(19) United States
(12) Patent Application Publication
(10) Pub. No.: US 2026/0092330 A1
(43) Pub. Date: Apr. 2, 2026

(54) ANALYTICAL METHODS, KIT AND DETECTION DEVICES

(71) Applicant: KABUSHIKI KAISHA TOSHIBA, Kawasaki-shi (JP)

(72) Inventors: Takuya MIYAGAWA, Yokohama (JP); Yoshitake SANO, Adachi (JP); Tomomi TAKAHASHI, Ota (JP); Mitsuko ISHIHARA, Setagaya (JP)

(73) Assignee: KABUSHIKI KAISHA TOSHIBA, Kawasaki-shi (JP)

(21) Appl. No.: 19/403,584

(22) Filed: Nov. 28, 2025

Related U.S. Application Data
(63) Continuation of application No. PCT/JP2024/007524, filed on Feb. 29, 2024.

(30) Foreign Application Priority Data
Jun. 20, 2023 (JP) 2023-100877

(51) Int. Cl. C12Q 1/6886

(52) U.S. Cl. CPC C12Q 1/6886

(57) ABSTRACT

According to one embodiment, analytical methods and kits are provided for determining the probability that a subject has breast cancer, pancreatic cancer, lung cancer, gastric cancer, or colorectal cancer. The analytical methods include quantifying a target miRNA selected from a group of target miRNAs in a sample derived from the subject to be analyzed.

### CROSS-REFERENCE TO RELATED APPLICATIONS

This application is a Continuation application of PCT Application No. PCT/JP2024/007524, filed Feb. 29, 2024 and based upon and claiming the benefit of priority from Japanese Patent Application No. 2023-100877, filed Jun. 20, 2023, the entire contents of all of which are incorporated herein by reference.

### REFERENCE TO A SEQUENCE LISTING

In accordance with 37 CFR § 1.831, the present specification makes reference to a Sequence Listing submitted electronically as a .xml file named “560625US.xml”. The .xml file was generated on Nov. 26, 2025 and is 168,687 bytes in size. The entire contents of the Sequence Listing are hereby incorporated by reference.

### FIELD

Embodiments described herein relate generally to analytical methods, kit and detection devices.

### BACKGROUND

Recently, the relationship between microRNA (miRNA) and diseases has attracted attention. The miRNA has a function to regulate a gene expression, and it is reported that the type and the amount of its expression changes from an early stage in various kinds of diseases. That is, in a patient with a certain disease, the amount of a particular miRNA increases or decreases compared to that of a normal person. Therefore, examining the amount of miRNA in a sample collected from a subject can be a measure of whether the subject patient has the particular disease.

### BRIEF DESCRIPTION OF DRAWINGS

FIG. 1 is a flowchart showing an example of an analysis method of a first embodiment, in which (a) is an example of an analysis method including a quantification step of any one of first target miRNAs, (b) is an example of an analysis method including a determination step of determining the probability of a subject having breast cancer, pancreatic cancer, lung cancer, gastric cancer, or colorectal cancer, (c) is an example of an analysis method including a determination step of determining prognosis or the presence of recurrence, and (d) is an example of an analysis method including a selection step of selecting the type of therapy or the type of drug to be applied to a subject.

FIG. 2 is a flowchart showing an example of an analysis method of a second embodiment, in which (a) is an example of an analysis method including a quantification step of any one of second target miRNAs, (b) is an example of an analysis method including a determination step of determining the probability of a subject having breast cancer, pancreatic cancer, lung cancer, gastric cancer, and colorectal cancer in a subject, (c) is an example of an analysis method including a determination step of determining prognosis or the presence of recurrence, and (d) is an example of an analysis method including a selection step of selecting the type of therapy or the type of drug to be applied to a subject.

FIG. 3A is a box plot showing experimental results of Example 1, showing quantitative values of hsa-miR-126-3p for each cancer type of sample.

FIG. 3B is a box plot showing experimental results of Example 1, showing quantitative values of hsa-miR-326 for each cancer type of sample.

FIG. 3C is a box plot showing experimental results of Example 1, showing quantitative values of hsa-miR-28-3p for each cancer type of sample.

FIG. 3D is a box plot showing experimental results of Example 1, showing quantitative values of hsa-miR-425-3p for each cancer type of sample.

FIG. 3E is a box plot showing experimental results of Example 1, showing quantitative values of hsa-miR-93-3p for each cancer type of sample.

FIG. 3F is a box plot showing experimental results of Example 1, showing quantitative values of hsa-miR-24-3p for each cancer type of sample.

FIG. 3G is a box plot showing experimental results of Example 1, showing quantitative values of hsa-miR-222-3p for each cancer type of sample.

FIG. 3H is a box plot showing experimental results of Example 1, showing quantitative values of hsa-miR-361-5p for each cancer type of sample.

FIG. 3I is a box plot showing experimental results of Example 1, showing quantitative values of hsa-miR-342-3p for each cancer type of sample.

FIG. 3J is a box plot showing experimental results of Example 1, showing quantitative values of hsa-miR-10b-5p for each cancer type of sample.

FIG. 4A is a box plot showing experimental results of Example 2, showing quantitative values of hsa-miR-128-3p for each cancer type of sample.

FIG. 4B is a box plot showing experimental results of Example 2, showing quantitative values of hsa-miR-151a-3p for each cancer type of sample.

FIG. 4C is a box plot showing experimental results of Example 2, showing quantitative values of hsa-miR-143-3p for each cancer type of sample.

FIG. 4D is a box plot showing experimental results of Example 2, showing quantitative values of hsa-miR-4770 for each cancer type of sample.

FIG. 4E is a box plot showing experimental results of Example 2, showing quantitative values of hsa-miR-1296-5p for each cancer type of sample.

FIG. 4F is a box plot showing experimental results of Example 2, showing quantitative values of hsa-miR-125a-5p for each cancer type of sample.

FIG. 4G is a box plot showing experimental results of Example 2, showing quantitative values of hsa-miR-409-3p for each cancer type of sample.

FIG. 4H is a box plot showing experimental results of Example 2, showing quantitative values of hsa-miR-99a-5p for each cancer type of sample.

FIG. 4I is a box plot showing experimental results of Example 2, showing quantitative values of hsa-miR-215-5p for each cancer type of sample.

### DETAILED DESCRIPTION

In general, according to one embodiment, an analytical method includes quantifying at least any one of hsa-miR-128-3p, hsa-miR-151a-3p, hsa-miR-143-3p, hsa-miR-4770, hsa-miR-1296-5p, hsa-miR-125a-5p, hsa-miR-409-3p, hsa-miR-99a-5p and hsa-miR-215-5p in a sample derived from a subject, and determining the probability of the subject having breast cancer, pancreatic cancer, lung cancer, gastric cancer or colorectal cancer.

Hereinafter, analytical methods, kit and detection device of the embodiments will be described with reference to the accompanying drawings.

### First Embodiment

### Analytical Method

An analytical method of the first embodiment includes quantifying at least any one of hsa-miR-128-3p, hsa-miR-151a-3p, hsa-miR-143-3p, hsa-miR-4770, hsa-miR-1296-5p, hsa-miR-125a-5p, hsa-miR-409-3p, hsa-miR-99a-5p and hsa-miR-215-5p in a sample derived from a subject, and determining the probability of the subject having breast cancer, pancreatic cancer, lung cancer, gastric cancer or colorectal cancer.

The total of 9 miRNAs described above are also referred to as “the first target miRNA group” in the following description (Hereinafter abbreviated and referred to simply as “target miRNA group” in some cases). The individual miRNA constituting the target miRNA group is also referred to as “the first target miRNA” (Hereinafter abbreviated and referred to simply as “target miRNA” in some cases.).

For example, each target miRNA is represented by a base sequence shown in Table 1 below. In the present specification, notation “T” on a sequence listing corresponding to each sequence number means “U”. The target miRNA to be quantified may be one type of the target miRNA group.

TABLE 1

| Sequence | Marker | |
|---|---|---|
| number | (Name of miRNA) | Base sequence |
| 1 | hsa-miR-128-3p | UCACAGUGAACCGGUCUCUUU |
| 2 | hsa-miR-151a-3p | CUAGACUGAAGCUCCUUGAGG |
| 3 | hsa-miR-143-3p | UGAGAUGAAGCACUGUAGCUC |
| 4 | hsa-miR-4770 | UGAGAUGACACUGUAGCU |
| 5 | hsa-miR-1296-5p | UUAGGGCCCUGGCUCCAUCUCC |
| 6 | hsa-miR-125a-5p | UCCCUGAGACCCUUUAACCUGUGA |
| 7 | hsa-miR-409-3p | GAAUGUUGCUCGGUGAACCCCU |
| 8 | hsa-miR-99a-5p | AACCCGUAGAUCCGAUCUUGUG |
| 9 | hsa-miR-215-5p | AUGACCUAUGAAUUGACAGAC |

The subject may be an animal to be subjected to the analysis, which is an animal providing a sample. The subject may be an animal contracting some kind of diseases or may be a normal animal. For example, the subject may be an animal which may be contracting cancer, an animal which may have been contracted cancer, or the like. In particular, the subject may be an animal possibly having at least any one of breast cancer, pancreatic cancer, lung cancer, gastric cancer, and colorectal cancer, an animal having at least any one of breast cancer, pancreatic cancer, lung cancer, gastric cancer, and colorectal cancer in the past, or the like.

The subject may preferably be a human, or the subject may be some other animal. Some other animal may be, for example, a mammal, such as a primate such as a monkey, a rodent such as a mouse, rat or guinea pig, a companion animal such as dog, cat or rabbit, or a domestic animal such as horse, cow or pigs, or a displayed animal or the like. In the case of an animal other than human, the target miRNA is a miRNA corresponding to the miRNA present in the animal.

The sample derived from a subject includes a sample collected from a subject or a sample obtained by appropriately treating the sample. A sample may preferably be serum or plasma. The sample may be some other body fluid, for example, blood, plasma, stroma liquid, urine, feces, sweat, saliva, oral mucosa, intranasal mucous membrane, pharyngeal mucosa, sputum, lymph fluid, cerebrospinal fluid, tears, mother's milk, amniotic fluid, semen or the like. Or, the sample may be cultured tissues or cells sampled from a subject, or a supernatant thereof.

The cancer according to the embodiment includes that of any stage of the disease, that is, for example, a state of remaining in an organ of an origin, a state of further extending to a surrounding tissue, a state of further metastasizing to a lymph node, a state of having metastasis to a further distant organ, and the like. For example, the breast cancer according to the embodiment refers to a malignant tumor (neoplasm) that begins to develop in mammary gland tissue. The breast cancer includes those generally referred to as “breast carcinoma” or “mammary cancer”. In addition, the various cancers in the present specification include, for example, an epithelial tumor, a non-epithelial tumor, or a tumor including both epithelial and non-epithelial tumors.

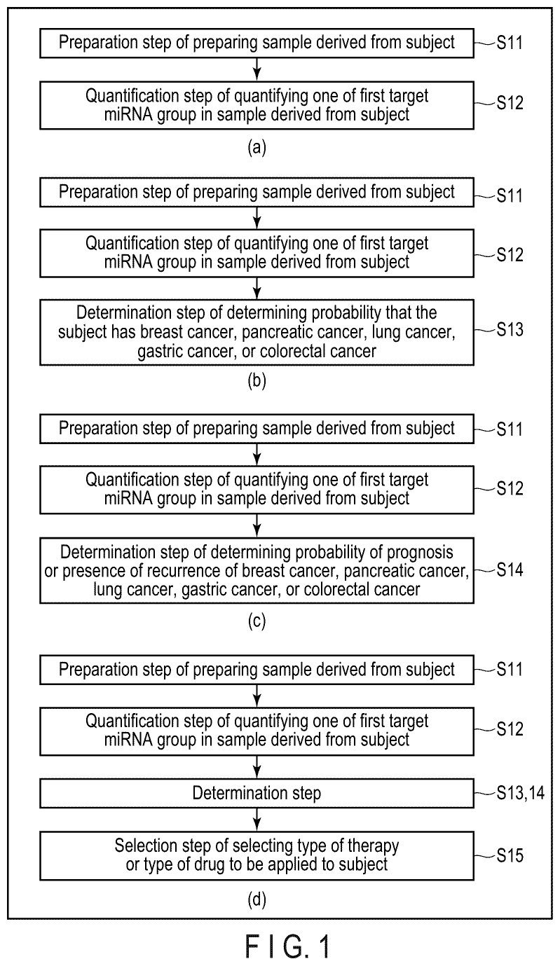

Hereinafter, an example of the procedure of the method of the first embodiment will be described with reference to (a), (b), and (c) of FIG. 1.

As shown in (a) of FIG. 1, the analysis method includes, for example, preparing a sample derived from a subject (preparation step (S11)); and quantifying one of the first target miRNA group in the sample derived from a subject (quantification step (S12)).

First, the sample derived from a subject is prepared (preparation step (S11)). The method of collecting a sample may be a general method based on the type of the sample. The collected sample may be, for example, pre-treated with any well-known means so as to set it, for example, to be in a condition not to inhibit a reverse transcription, elongation and amplification reactions, which will be described below or to set it in a condition more suitable for these reactions. The pre-treatment is, for example, slicing, homogenizing, centrifuging, precipitation, extraction and/or separation or the like.

For example, the extraction may be carried out with use of a commercially available nucleic acid extraction kit. As the nucleic acid extraction kit, for example, NucleoSpin (registered trademark) miRNA Plasma (manufactured by Takara Bio Inc.), Quick-cfRNA Serum & Plasma Kit (manufactured by Zymo Research Corporation), miRNeasy Serum/Plasma kit (manufactured by Qiagen), miRVana (registered trademark) PARIS isolation kit (manufactured by Thermo Fisher Scientific Inc.), PureLink (registered trademark) Total RNA Blood Kit (manufactured by Thermo Fisher Scientific Inc.), Plasma/Serum RNA Purification Kit (manufactured by Norgen Biotek Corp.), microRNA Extractor (registered trademark) SP Kit (manufactured by FUJIFILM Wako Pure Chemical Corporation), High Pure miRNA Isolation Kit (manufactured by Sigma-Aldrich), or the like can be used. Or, the extraction may be carried out without using a commercially available kit, but by, for example, diluting the material with a buffer solution, subjecting it to a heat-treatment at 80 to 100° C. and centrifuging, and then collecting its supernatant.

Next, one of the first target miRNA group contained in the sample derived from a subject is quantified (quantification step (S12)). The quantification step (S12) can be performed using a general method for quantifying RNA, particularly short-chain RNA such as miRNA. Examples of the general method include a method of reverse-transcribing a target miRNA to generate cDNA, amplifying the obtained cDNA, and detecting and quantifying an amplification product. When the RNA is a short chain, in order to facilitate amplification, it is also common practice to elongate the short RNA so that artificial sequences are added to the ends of the short RNA and then reverse transcribe, or to elongate the reverse transcribed cDNA so that artificial sequences are added to the ends. In addition, a rolling circle amplification method is known as a technique for directly amplifying RNA in a sample without performing reverse transcription, and detecting and quantifying an amplification product. Furthermore, when the concentration of the target miRNA in the sample is relatively high or when a device capable of measuring high sensitivity can be used, directly detecting the target miRNA (or cDNA thereof) without amplifying the target miRNA is also one of general methods. Examples of the device capable of direct detection include a microarray including a nucleic acid probe that specifically binds to the target miRNA.

For the amplification, for example, a PCR method (including a qPCR method) or a LAMP method can be used. Detection and quantification may be performed after amplification or over time during amplification. For the detection and quantification, for example, a measurement method using a signal based on turbidity or absorbance, a measurement method using an optical signal, a measurement method using an electrochemical signal, a combination thereof or the like can be used. For example, the target miRNA can be quantified from the intensity or the amount of change of the signal correlated with the amount of the amplified product, the time (rise time) until the signal reaches the threshold value, or the number of rise cycles when the PCR method is used. For the detection and quantification, for example, a result of a next generation sequencing (NGS) method may be used. In that case, the target miRNA can be relatively quantified from the detection result such as the number of reads aligned with the target miRNA.

The quantitative value of target miRNA may be determined using a calibration curve representing a relationship between the detection result of the signal and the abundance of target miRNA. The calibration curve can be created by detecting signals for a plurality of standard samples containing target miRNAs at different known concentrations. The abundance of target miRNA in the sample can be calculated by comparing the calibration curve with the detection result of the signal obtained for the sample derived from a subject. The abundance of target miRNA in the sample may be calculated, for example, as the number of copies of the target miRNA per unit amount of the sample.

The quantification in the quantification step (S12) may be performed using, for example, a commercially available kit. Examples of the commercially available kit include TaqMan (registered trademark) Advanced miRNA Assays (manufactured by Thermo Fisher Scientific Inc., catalog No. A25576) and miRCURY LNA (registered trademark) miRNA PCR Assays (manufactured by Qiagen, catalog No. 339306), SYBR (registered trademark) Green qPCR microRNA detection system (manufactured by Origin Technologies, Inc.), and the kit can be used together with a primer or a probe that specifically amplifies the target miRNA.

The data relating to the detection of the first target miRNA obtained in the quantification step (S12) can be used for determining the probability of the subject having breast cancer, pancreatic cancer, lung cancer, gastric cancer or colorectal cancer. For example, as shown in (b) of FIG. 1, the analysis method of the first embodiment may further include a step (S13) of determining the probability of the subject having breast cancer, pancreatic cancer, lung cancer, gastric cancer or colorectal cancer, which is performed after the quantification step (S12).

It should be note that “determining the probability of the subject having breast cancer, pancreatic cancer, lung cancer, gastric cancer or colorectal cancer” means that subjects are susceptible to all five types of cancer, but the type of cancer the subject has is not specified as any one of the five types of cancer, or the subject is not determined to have any of the five types of cancer.

In other words, “subjects are susceptible to all five types of cancer, but the type of cancer the subject has is not specified as any one of the five types of cancer” means that determining the presence of morbidity of at least any one of breast cancer, pancreatic cancer, lung cancer, gastric cancer, and colorectal cancer in the subject.

In other words, by “determining the probability of the subject having breast cancer, pancreatic cancer, lung cancer, gastric cancer or colorectal cancer”, the possibility that the subject has breast cancer, the possibility that the subject has pancreatic cancer, the possibility that the subject has lung cancer, the possibility that the subject has gastric cancer, and the possibility that the subject has colorectal cancer are presented simultaneously, or the possibility that the subject has none of the breast, pancreatic, lung, gastric and colorectal cancer is presented.

That is, this method intends to present as many possible cancer types as possible without missing any possibility of cancer, as primary screening in cancer checkups, in view of the importance of early detection and early treatment of cancer.

In the determination step (S13), it is possible to provide information for assisting determination for the probability of the subject having breast cancer, pancreatic cancer, lung cancer, gastric cancer or colorectal cancer.

For example, the determination step (S13) is performed based on the quantitative value of target miRNA in the sample derived from a control obtained by performing quantification on the sample derived from a control in parallel with the quantification step (S12) on the sample derived from a subject. That is, the method of the first embodiment includes quantifying miRNA in the sample derived from a control; comparing the quantitative value of the first target miRNA in the subject with the quantitative value of the first target miRNA in the control to determine the probability of the subject having breast cancer, pancreatic cancer, lung cancer, gastric cancer or colorectal cancer.

The quantitative value of the first target miRNA in the control is a quantitative value of target miRNA obtained in advance using the same method as used in the quantification step (S12), for example, for a sample same as or similar to the sample derived from a subject (for example, when the sample derived from a subject is serum, the sample derived from a control is serum or plasma). A plurality of samples corresponding to a control may be prepared, and determination may be performed based on a numerical range including values obtained by quantifying each of the plurality of samples.

The control may be, for example, a healthy individual. The healthy individual refers to an individual who does not suffer from at least cancer. The healthy individual is preferably a healthy individual who does not have a disease or abnormality.

The individual selected as a control may be an individual different from the subject to be analyzed by the present method, but is preferably an individual belonging to the same type of cancer, that is, a human if the subject is a human. In addition, physical conditions such as age, sex, height, and weight, or the number of persons of the control are not particularly limited, but the physical conditions are preferably the same as or similar to those of the subject to be tested by the present analysis method.

Alternatively, the determination step (S13) may be performed based on a preset threshold value or the like. The threshold value is, for example, an abundance of first target miRNA that can separate a quantitative value in a sample known to suffer from at least any one of breast cancer, pancreatic cancer, lung cancer, gastric cancer, and colorectal cancer (hereinafter, referred to as “standard sample”) from a quantitative value in a healthy individual. The standard sample is, for example, a sample of another site derived from a subject, a sample derived from an individual similar to (for example, the same type as) the subject, or a sample containing established cells of at least any one of breast cancer, pancreatic cancer, lung cancer, gastric cancer, and colorectal cancer. The quantitative value in the standard sample is preferably obtained by applying the same method as the quantification step (S12) of the subject. The value for separating the quantitative value in the standard sample and the quantitative value in the healthy individual may be determined based on statistical criteria, the maximum value or the minimum value of the quantitative value in the standard sample may be used as the threshold value, or the maximum value or the minimum value of the quantitative value in the control may be used as the threshold value. Furthermore, the threshold value may be determined according to the quantification method, type of sample and measurement conditions used, and the like.

Alternatively, the threshold value may be determined for each subject. For example, if the quantitative value of first target miRNA in a healthy state of the subject is monitored (for example, periodic medical examination or the like), by using the quantitative value as the threshold value, it is possible to issue an alarm indicating that there is probability of suffering from breast cancer, pancreatic cancer, lung cancer, gastric cancer or colorectal cancer in the subject when the quantitative value is higher or lower than the threshold value. The threshold value may vary from individual to individual. For example, in subject A in which the quantitative value of miRNA has been usually remained at about 10³ copies, once the quantitative value of target miRNA is 10⁴ copies, it can be determined that there is all probability of suffering from breast cancer, pancreatic cancer, lung cancer, gastric cancer or colorectal cancer in the subject. On the other hand, in the subject B in which the quantitative value of miRNA has been remained at about 10² copies, once the quantitative value of target miRNA is 10³ copies, it can be determined that there is all probability of suffering from breast cancer, pancreatic cancer, lung cancer, gastric cancer or colorectal cancer in the subject.

Here, the quantitative value or threshold value in the control, which serves as a criterion for determination, may be determined from past knowledge such as literature. Determination of morbidity also includes determination of a high possibility of morbidity. Conversely, determination of no morbidity also includes determination of a low possibility of morbidity.

In a subject suffering from breast cancer, pancreatic cancer, lung cancer, gastric cancer, or colorectal cancer, the target miRNA group includes those having a higher expression level (high expression target miRNA) and those having a lower expression level (low expression target miRNA) than the control. When the quantitative value of the low expression target miRNA is lower than that of the control, all probability of suffering from breast cancer, pancreatic cancer, lung cancer, gastric cancer or colorectal cancer in the subject can be determined. On the other hand, when the quantitative value of the high expression target miRNA is higher than that of the control, all probability of suffering from breast cancer, pancreatic cancer, lung cancer, gastric cancer or colorectal cancer in the subject can be determined.

As used herein, the phrase “determining the probability of the subject having breast cancer, pancreatic cancer, lung cancer, gastric cancer or colorectal cancer” includes that whether it is determined to have breast cancer, pancreatic cancer, lung cancer, gastric cancer or colorectal cancer, whether it is determined to have breast cancer, pancreatic cancer, lung cancer, or colorectal cancer, whether it is determined to have breast cancer, pancreatic cancer, or gastric cancer, whether it is determined to have breast cancer, or pancreatic cancer.

In detail, each of hsa-miR-128-3p (SEQ ID NO:1), hsa-miR-151a-3p (SEQ ID NO:2), hsa-miR-4770 (SEQ ID NO:4), hsa-miR-125a-5p (SEQ ID NO:6), hsa-miR-409-3p (SEQ ID NO:7) and hsa-miR-215-5p (SEQ ID NO:9) can determine that the subject has breast cancer, pancreatic cancer, lung cancer, gastric cancer or colorectal cancer when the control is a healthy individual and the quantitative value of the subject is smaller than the quantitative value of the control. hsa-miR-143-3p (SEQ ID NO:3) or hsa-miR-1296-5p (SEQ ID NO:5) can determine that the subject has breast cancer, pancreatic cancer, lung cancer, or gastric cancer when the control is a healthy individual and the quantitative value of the subject is smaller than the quantitative value of the control. hsa-miR-99a-5p (SEQ ID NO:8) can determine that the subject has breast cancer, pancreatic cancer, lung cancer, or colorectal cancer when the control is a healthy individual and the quantitative value of the subject is smaller than the quantitative value of the control.

The difference between the quantitative value of each miRNA in the control and the quantitative value of each miRNA in the subject is preferably statistically significant. Whether or not it is statistically significant can be determined by preparing a plurality of samples known to suffer from various cancers, confirming the possible numerical range of the quantitative value of each miRNA in the sample, and calculating the probability distribution in advance or calculating the probability distribution from information such as known literature.

According to a further embodiment, determining the probability of having breast cancer, pancreatic cancer, lung cancer, gastric cancer, or colorectal cancer includes determining prognosis or recurrence of breast cancer, pancreatic cancer, lung cancer, gastric cancer, or colorectal cancer in the subject. For example, as shown in (c) of FIG. 1, the analysis method includes, after the quantification step (S12), a prognosis and recurrence determination step (S14) of determining prognosis or the presence of recurrence of breast cancer, pancreatic cancer, lung cancer, gastric cancer, or colorectal cancer in the subject from the result of quantification. In the prognosis and recurrence determination step (S14), for example, when the quantitative value is high or low, it is possible to determine that the prognosis of breast cancer, pancreatic cancer, lung cancer, gastric cancer, or colorectal cancer in the subject is poor, or at least any one of breast cancer, pancreatic cancer, lung cancer, gastric cancer, or colorectal cancer has recurred, or the possibility of recurrence is high. The quantitative value and threshold value in the control can also be used for determination of prognosis and recurrence. In particular, it may be preferable to use a threshold value determined for each subject.

In addition, after the determination step (S13) and/or the prognosis and recurrence determination step (S14), it is also possible to select the type of therapy or the type of drug to be applied to the subject according to the determination result and to assist the selection. For example, as shown in (d) of FIG. 1, the analysis method includes, after the determination step (S13) and/or the prognosis and recurrence determination step (S14), a selection step (S15) of selecting the type of therapy or the type of drug to be applied to the subject from the determination result. Here, the therapy or drug is for the treatment of breast cancer, pancreatic cancer, lung cancer, gastric cancer or colorectal cancer. Determining the type of therapy or the type of drug includes determining the used amount, timing or duration of the therapy or drug.

According to the analysis method of the first embodiment described above, it is possible to easily determine the probability of a subject having breast cancer, pancreatic cancer, lung cancer, gastric cancer or colorectal cancer by quantifying one target miRNA out of the first target miRNA group in the sample derived from a subject and comparing the quantitative value with a quantitative value of the miRNA in the sample derived from a control.

Since the method of the present embodiment can use serum or plasma that can be easily collected in a medical examination or the like, morbidity of breast cancer, pancreatic cancer, lung cancer, gastric cancer, or colorectal cancer can be found at an early stage. By using serum, plasma, or the like, the physical and economic burden on the subject can be greatly reduced as compared with cytodiagnosis or the like, and the procedure is easy, so that the burden on an examiner is also small. In addition, since the concentration of miRNA contained in serum or plasma is stable, it is possible to perform a more accurate test.

According to a further embodiment, there is also provided an analysis method for assisting determination of the probability of a subject having breast cancer, pancreatic cancer, lung cancer, gastric cancer, or colorectal cancer, the analysis method including quantifying a first target miRNA in the sample derived from a subject (quantification step (S12)). The phrase “assisting determination” includes, for example, acquiring information about the possibility of the subject having breast cancer, pancreatic cancer, lung cancer, gastric cancer, or colorectal cancer. The term “information” is, for example, information about an analysis result of a sample, and can be, for example, a quantitative value. According to the present method, it is possible to acquire more accurate information for determining the probability of the subject having breast cancer, pancreatic cancer, lung cancer, gastric cancer, or colorectal cancer, determining prognosis, determining the presence of recurrence, selecting the therapy or drug to be applied to the subject, or the like.

According to a further embodiment, the control may be, for example, an individual confirmed to have breast cancer, pancreatic cancer, lung cancer, gastric cancer, or colorectal cancer by examination, or the like. That is, the control is an individual known to have breast cancer, pancreatic cancer, lung cancer, gastric cancer, or colorectal cancer. That is, in such a determination step (S13), it is determined whether the subject have the cancer type that the control has. Specifically, the determination is made by quantifying a biomarker in the sample derived from a subject with reference to a quantitative value of the biomarker indicating that the subject suffers from a specific cancer in the sample derived from a control, and comparing the biomarker with the reference.

In detail, each of the hsa-miR-128-3p (SEQ ID NO:1), hsa-miR-151a-3p (SEQ ID NO:2), hsa-miR-143-3p (SEQ ID NO:3) and hsa-miR-1296-5p (SEQ ID NO:5) can determine that the subject suffers from breast cancer, pancreatic cancer, lung cancer or gastric cancer when the control is a colorectal cancer subject and the quantitative value of the subject is smaller than the quantitative value of the control. hsa-miR-4770 (SEQ ID NO:4) can determine that the subject suffers from breast cancer, pancreatic cancer or gastric cancer when the control is a colorectal cancer subject and the quantitative value of the subject is smaller than the quantitative value of the control. hsa-miR-409-3p (SEQ ID NO:7) can determine that the subject suffers from breast cancer or pancreatic cancer when the control is a colorectal cancer subject and the quantitative value of the subject is smaller than the quantitative value of the control.

Some of the miRNAs in the first target miRNA group can also be used as markers to identify to one cancer type by setting the control to a specific cancer type. In other words, some miRNAs in the first group of miRNAs can be used for purposes other than determining “there is probability of suffering from breast cancer, pancreatic cancer, lung cancer, gastric cancer or colorectal cancer in the subject”. For example, it can be determined that a subject has breast cancer.

Some miRNAs of the first target miRNA group are, for example hsa-miR-128-3p (SEQ ID NO:1), hsa-miR-151a-3p (SEQ ID NO:2), hsa-miR-143-3p (SEQ ID NO:3), hsa-miR-4770 (SEQ ID NO:4), hsa-miR-125a-5p (SEQ ID NO:6), hsa-miR-99a-5p (SEQ ID NO:8). hsa-miR-128-3p (SEQ ID NO:1) can determine that the subject suffers from breast cancer when the control is individuals having lung cancer or gastric cancer. Each of hsa-miR-151a-3p (SEQ ID NO:2), hsa-miR-143-3p (SEQ ID NO:3) and hsa-miR-4770 (SEQ ID NO:4) can determine that the subject suffers from breast cancer when the control is individuals having lung cancer. hsa-miR-125a-5p (SEQ ID NO:6) can determine that the subject suffers from breast cancer when the control is individuals having colorectal cancer, lung cancer or gastric cancer. hsa-miR-99a-5p (SEQ ID NO:8) can determine that the subject suffers from breast cancer when the control is individuals having gastric cancer.

According to a further embodiment, the present analysis method can also be used for detection of breast cancer cells, pancreatic cancer cells, lung cancer cells, gastric cancer cells or colorectal cancer cells in a sample not derived from a subject, and the like. For example, in the case of artificially producing breast cancer cells, pancreatic cancer cells, lung cancer cells, gastric cancer cells or colorectal cancer cells, it can also be used when confirming whether the same cells are present in the produced cell-containing solution, and the like.

### (Marker)

According to the first embodiment, there is provided a marker for detecting at least any one of breast cancer, pancreatic cancer, lung cancer, gastric cancer, and colorectal cancer of the subject, the marker including one target miRNA.

Here, the “marker” refers to a substance capable of determining whether a sample and/or a subject from which the sample is derived is in a specific state by detecting its presence or concentration in the sample.

As the marker of the first embodiment for detecting the probability of having breast cancer, pancreatic cancer, lung cancer, gastric cancer, and colorectal cancer, for example, by measuring the abundance (quantitative value) of the marker in the sample derived from a subject, it is possible to determine the probability of having the subject breast cancer, pancreatic cancer, lung cancer, gastric cancer, and colorectal cancer, determine prognosis or the presence of recurrence, select the therapy or drug to be applied to the subject, or the like, as described above.

### (Kit)

According to the first embodiment, a kit for detecting the probability of having breast cancer, pancreatic cancer, lung cancer, gastric cancer, and colorectal cancer is provided.

The kit contains reagents that can be used in a general method for quantifying RNA, particularly short-chain RNA such as miRNA, and a nucleic acid capable of specifically binding to a target miRNA (that is, it hybridizes with the target miRNA). When a qPCR method is used for detection of the target miRNA, the nucleic acid capable of specifically binding to the target miRNA may be a reverse transcription (RT) primer for reverse transcription of the target miRNA, an elongation (EL) primer for elongation of the target miRNA, or an amplification primer set for amplification of the target miRNA.

The RT primer is a primer for obtaining cDNA of the target miRNA. The RT primer includes a sequence complementary to at least a portion of the sequence of the target miRNA. The RT primer may further include an artificial sequence added to the cDNA to facilitate amplification of the cDNA of the target miRNA.

The EL primer is a primer for adding an artificial sequence to the cDNA of the target miRNA to facilitate amplification of the cDNA. The EL primer may include a sequence complementary to at least a portion of the sequence of the cDNA of the target miRNA and a sequence added for elongation of each cDNA.

The amplification primer set contains at least a forward primer and a reverse primer, for example, for PCR method. Alternatively, the amplification primer set may be for LAMP method, and may contain primers of a sequence corresponding to the base sequence of the target miRNA used in the general LAMP method. Alternatively, the amplification primer set may be for NGS method, for example, and may contain a forward primer containing an artificial adaptor sequence and a reverse primer containing a complementary sequence thereof. The amplification primer set for NGS method may contain a plurality of types of combinations of forward primers and reverse primers including different barcode sequences, in order to simultaneously analyze a plurality of samples. When the amplification primer set is used in rolling cycle amplification method, the kit further contains a circular single-stranded DNA that is hybridized by the amplification primer and serves as a template for amplification.

Each primer contained in the amplification primer set may be designed to bind to cDNA of the target miRNA or a complementary sequence thereof, or may be designed to bind to an artificial sequence added by an RT primer and/or an EL primer.

Nucleic acid probes designed to bind to the target cDNA or its complementary sequence may be used to detect the amplifying cDNA.

Further, when the target miRNA in the sample is directly detected by a microarray, the nucleic acid capable of specifically binding to the target miRNA is a nucleic acid probe included in the microarray. The nucleic acid probe may have at least a portion of, or a complementary sequence of, the sequence of the target miRNA, cDNA thereof, or an amplification product thereof.

The nucleic acid contained in the kit may be provided by being stored in a container together with an appropriate carrier individually or in combination. The appropriate carrier is, for example, water, a physiological solution or a buffer. The container is, for example, a tube or a microtiter plate. Alternatively, these nucleic acids may be provided by being immobilized on a solid phase such as a microfluidic chip.

In addition to the nucleic acid, the kit may contain a reagent used for reverse transcription, elongation or amplification, for example, an enzyme, a substrate, and/or a labeling substance that generates an optical signal or an electrochemical signal used for detection, and the like. The labeling substance is, for example, a fluorescent dye such as SYBR (registered trademark) Green, EvaGreen (registered trademark), or SYTO (registered trademark) 82, or an indicator such as a metal complex such as ruthenium hexamine in the case of current detection.

The kit can be used, for example, for determining the probability of the subject having breast cancer, pancreatic cancer, lung cancer, gastric cancer, and colorectal cancer, determining prognosis, determining the presence of recurrence, selecting the type of therapy or the type of drug or the like.

According to a further embodiment, a kit for detecting the probability of having breast cancer, pancreatic cancer, lung cancer, gastric cancer, and colorectal cancer is provided as a composition or a diagnostic agent for determining the probability of having breast cancer, pancreatic cancer, lung cancer, gastric cancer, and colorectal cancer. Further, according to the embodiment, there is also provided a use of at least any one of the nucleic acids in the production of a composition or a diagnostic agent for determining the probability of having breast cancer, pancreatic cancer, lung cancer, gastric cancer, and colorectal cancer.

### Second Embodiment

### Analysis Method

An analysis method according to a second embodiment includes quantifying at least any one of hsa-miR-126-3p, hsa-miR-326, hsa-miR-28-3p, hsa-miR-425-3p, hsa-miR-93-3p, hsa-miR-24-3p, hsa-miR-222-3p, hsa-miR-361-5p and hsa-miR-342-3p in a sample derived from a subject, and determining the probability of having breast cancer, pancreatic cancer or colorectal cancer.

The target miRNAs of the second embodiment (hereinafter referred to as “second target miRNAs”) are each represented by the sequences shown in Table 2 below. In other words, the target miRNA to be quantified in the second embodiment should be one of the second target miRNA group, as shown in Table 2.

TABLE 2

| Sequence | Marker | |
|---|---|---|
| number | (Name of miRNA) | Base sequence |
| 10 | hsa-miR-126-3p | UCGUACCGUGAGUAAUAAUGCG |
| 11 | hsa-miR-326 | CCUCUGGGCCCUUCCUCCAG |
| 12 | hsa-miR-28-3p | CACUAGAUUGUGAGCUCCUGGA |
| 13 | hsa-miR-425-3p | AUCGGGAAUGUCGUGUCCGCCC |
| 14 | hsa-miR-93-3p | ACUGCUGAGCUAGCACUUCCCG |
| 15 | hsa-miR-24-3p | UGGCUCAGUUCAGCAGGAACAG |
| 16 | hsa-miR-222-3p | AGCUACAUCUGGCUACUGGGU |
| 17 | hsa-miR-361-5p | UUAUCAGAAUCUCCAGGGGUAC |
| 18 | hsa-miR-342-3p | UCUCACACAGAAAUCGCACCCGU |

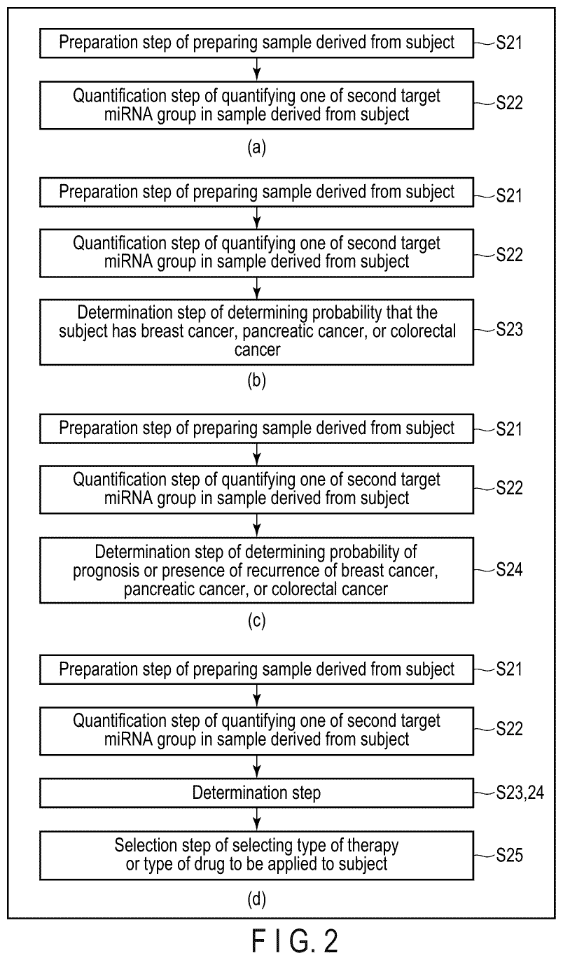

As shown in of (a) of FIG. 2, the method of the second embodiment includes, for example, preparing a sample derived from a subject (preparation step (S21)); and quantifying any one types of second target miRNAs group in the sample derived from a subject (quantification step (S22)).

The data from detection of target miRNAs obtained in the quantification step (S22) can be used for determining the probability of the subject having breast cancer, pancreatic cancer, or colorectal cancer. For example, the analysis method of the second embodiment can further include a determination step (S23) for the probability of having breast cancer, pancreatic cancer or colorectal cancer that can be performed after the quantification step (S22).

In the determination step (S23), for example, a criterion is set for each second target miRNA, and determination is made by comparing the criterion with the quantitative value obtained in the quantification step (S22). The criterion of each target miRNA of the subject is preferably a quantitative value, a threshold value, or the like of the corresponding target miRNA of the control, and each threshold value is preferably determined for each type of target miRNA. That is, the method of the second embodiment includes quantifying target miRNA in a sample derived from a control; and comparing the quantitative values of target miRNAs in the subject with the quantitative values of the target miRNAs in the control to determine the probability of having breast cancer, pancreatic cancer, or colorectal cancer, similar to that of the first embodiment.

The control is a healthy individual or an individual known to have breast cancer, pancreatic cancer, lung cancer, gastric cancer, or colorectal cancer.

When the control has breast cancer, pancreatic cancer or colorectal cancer, in such a determination step (S23), whether or not the subject has the cancer type from which the control has is determined. Specifically, the determination is made by quantifying a biomarker in the sample derived from a subject, and comparing the biomarker of the subject with reference to a quantitative value of the biomarker in the sample derived from a control. The reference indicates that the control suffers from a specific cancer.

Note that “determining the possibility of the subject having breast cancer, pancreatic cancer and colorectal cancer” means that the subject having the possibility of the subject having three types of cancers but the type of cancer has is not specified as any one of the three types of cancer, or the subject is not determined to have any of the three types of cancer.

“The subject having the possibility of the subject having three types of cancers but the type of cancer has is not specified as any one of the three types of cancer” means that determining morbidity of at least any one of breast cancer, pancreatic cancer and colorectal cancer in the subject.

In other words, by “determining the possibility of the subject having breast cancer, pancreatic cancer and colorectal cancer”, the possibility that the subject has breast cancer, the possibility that the subject has pancreatic cancer, and the possibility that the subject has colorectal cancer are presented simultaneously, or the possibility that the subject has none of the breast, pancreatic and colorectal cancer is presented.

That is, this method intends to present as many possible cancer types as possible without missing any possibility of cancer, as primary screening in cancer checkups, in view of the importance of early detection and early treatment of cancer.

As used herein, the phrase “determining the probability of having the subject breast cancer, pancreatic cancer, or colorectal cancer” includes that whether it is determined to have breast cancer, pancreatic cancer or colorectal cancer, whether it is determined to have pancreatic cancer, or colorectal cancer.

In detail, each of hsa-miR-126-3p (SEQ ID NO:10), hsa-miR-326 (SEQ ID NO:11), hsa-miR-28-3p (SEQ ID NO:12), hsa-miR-425-3p (SEQ ID NO:13), hsa-miR-93-3p (SEQ ID NO:14), hsa-miR-24-3p (SEQ ID NO:15), hsa-miR-222-3p (SEQ ID NO:16) and hsa-miR-361-5p (SEQ ID NO:17) can determine that the subject has breast cancer, pancreatic cancer or colorectal cancer when the control is a healthy individual and the quantitative value of the subject is smaller than the quantitative value of the control. hsa-miR-342-3p (SEQ ID NO:18) can determine that the subject suffers from pancreatic cancer or colorectal cancer when the control is a healthy individual or breast cancer, and the quantitative value of the subject is smaller than the quantitative value of the control.

In addition, some of the miRNAs in the second target miRNA group can be used as markers to identify to one cancer type by using a specific group of samples as controls. That is, some of the miRNAs in the second target miRNA group could be used to make a determination other than the determination of “the probability of the subject having breast, pancreatic, and colorectal cancers”. For example, it can be determined that the subject has colorectal cancer.

Some miRNAs in the second target miRNA group mentioned above are, specifically, hsa-miR-126-3p (SEQ ID NO:10), hsa-miR-326 (SEQ ID NO:11), hsa-miR-28-3p (SEQ ID NO:12), hsa-miR-425-3p (SEQ ID NO:13), hsa-miR-93-3p (SEQ ID NO:14), hsa-miR-24-3p (SEQ ID NO:15), hsa-miR-222-3p (SEQ ID NO:16), hsa-miR-361-5p (SEQ ID NO:17) and hsa-miR-342-3p (SEQ ID NO:18). In addition, hsa-miR-10b-5p (SEQ ID NO:19, UACCCUGUAGAACCGAAUUUGUG) can also be used as a marker to identify to one cancer type.

Each of hsa-miR-126-3p (SEQ ID NO:10), hsa-miR-326 (SEQ ID NO:11), hsa-miR-28-3p (SEQ ID NO:12), hsa-miR-425-3p (SEQ ID NO:13), hsa-miR-93-3p (SEQ ID NO:14), hsa-miR-24-3p (SEQ ID NO:15), hsa-miR-222-3p (SEQ ID NO:16) and hsa-miR-361-5p (SEQ ID NO:17) can determine that the subject has colorectal cancer when the control is breast cancer or pancreatic cancer and the quantitative value of the subject is smaller than the quantitative value of the control. hsa-miR-342-3p (SEQ ID NO:18) can determine that the subject has colorectal cancer when the control is pancreatic cancer and the quantitative value of the subject is smaller than the quantitative value of the control. hsa-miR-10b-5p (SEQ ID NO:19) can determine that the subject has colorectal cancer when the control is healthy individual, breast cancer or pancreatic cancer and the quantitative value of the subject is smaller than the quantitative value of the control.

According to a further embodiment, determining the probability of the subject having breast cancer, pancreatic cancer or colorectal cancer includes determining prognosis or recurrence of breast cancer, pancreatic cancer or colorectal cancer. For example, as shown in (c) of FIG. 2, the analysis method includes, after the quantification step (S22), a prognosis and recurrence determination step (S24) of determining prognosis or the presence of recurrence of breast cancer, pancreatic cancer or colorectal cancer in the subject from the result of quantification. In the prognosis and recurrence determination step (S14), for example, when the quantitative value is high or low, it is possible to determine that the prognosis of breast cancer, pancreatic cancer or colorectal cancer in the subject is poor, or at least any one of breast cancer, pancreatic cancer or colorectal cancer has recurred, or the possibility of recurrence is high. The quantitative value and threshold value in the control can also be used for determination of prognosis and recurrence. In particular, it may be preferable to use a threshold value determined for each subject.

In addition, after the determination step (S23) and/or the prognosis and recurrence determination step (S24), it is also possible to select the type of therapy or the type of drug to be applied to the subject according to the determination result and to assist the selection. For example, as shown in (d) of FIG. 2, the analysis method includes, after the determination step (S23) and/or the prognosis and recurrence determination step (S24), a selection step (S25) of selecting the type of therapy or the type of drug to be applied to the subject from the determination result. Here, the therapy or drug is for the treatment of breast cancer, pancreatic cancer or colorectal cancer. Determining the type of therapy or the type of drug includes determining the used amount, timing or duration of the therapy or drug.

According to the analysis method of the second embodiment described above, it is possible to easily determine the probability of a subject having breast cancer, pancreatic cancer or colorectal cancer by quantifying one target miRNA out of the second target miRNA group in the sample derived from a subject and comparing the quantitative value with a quantitative value of the miRNA in the sample derived from a control.

According to a further embodiment, the present analysis method can also be used for detection of breast cancer cells, pancreatic cancer cells or colorectal cancer cells in a sample not derived from a subject, and the like. For example, in the case of artificially producing breast cancer cells, pancreatic cancer cells or colorectal cancer cells, it can also be used when confirming whether or not the same cells are present in the produced cell-containing solution, and the like.

According to the second embodiment, there is provided a marker for detecting at least any one of breast cancer, pancreatic cancer and colorectal cancer of the subject, the marker including one type of the second target miRNA.

As the marker of the second embodiment for detecting the probability of having breast cancer, pancreatic cancer and colorectal cancer, for example, by measuring the abundance (quantitative value) of the marker in the sample derived from a subject, it is possible to determine the probability of the subject having breast cancer, pancreatic cancer and colorectal cancer, determine prognosis or the presence of recurrence, select the therapy or drug to be applied to the subject, or the like, as described above.

According to the second embodiment, a kit for detecting the probability of having breast cancer, pancreatic cancer and colorectal cancer is provided.

The kit of the second embodiment contains reagents that can be used in a general method for quantifying RNA, particularly short-chain RNA such as miRNA, and a nucleic acid capable of specifically binding to a target miRNA, similar to that of the first embodiment.

The kit can be used, for example, for determining the probability of the subject having breast cancer, pancreatic cancer or colorectal cancer, determining prognosis, determining the presence of recurrence, selecting the type of therapy or the type of drug or the like.

According to a further embodiment, a kit for detecting the probability of having breast cancer, pancreatic cancer and colorectal cancer is provided as a composition or a diagnostic agent for determining the probability of having breast cancer, pancreatic cancer and colorectal cancer. Further, according to the embodiment, there is also provided a use of at least any one of the nucleic acids in the production of the composition or the diagnostic agent.

### EXAMPLES

Experiments carried out using the analytical methods or the kits of the embodiments will now be described.

### Example 1. Search and Selection of miRNA Markers for Distinguishing Between Various Cancer Individuals and Healthy Individuals

Some miRNA markers capable of distinguishing breast Cancer individuals, pancreatic Cancer individuals, and colorectal Cancer individuals from healthy individuals were searched as follows.

### Preparation of Samples

As samples, breast cancer patient sera, pancreatic cancer patient sera, colorectal cancer patient sera, and healthy individual sera were prepared for 24 samples each, for a total of 96 samples.

Treatment of Samples and Quantification of miRNAs

From each serum, RNA was extracted. The extraction was carried out using NucleoSpin (registered trademark) miRNA Plasma (a product of TAKARA BIO Corporation).

For synthesis of cDNA, TaqMan miRNA cDNA Synthesis Kit (Applied Biosystems, Cat. A28007) was used. The TaaMan miRNA cDNA Synthesis Kit is a kit in which addition of a poly (A) chain to the 3′ end of mature RNA and ligation of an adaptor sequence to the 5′ end are performed, whereby not target-specific but all mature RNAs present in a sample are generally reverse-transcribed. Therefore, cDNAs corresponding to all mature RNAs including miRNA in each sample were obtained.

miRNAs among the obtained cDNAs were quantified by performing RT-qPCR using TaqMan Fast Advanced Master Mix (manufactured by Applied Biosystems) and TaqMan Advanced miRNA Assays (manufactured by Applied Biosystems) according to the protocol included in the package. The TaqMan PCR method, which is one of 5′ nuclease assays, is an amplification method characterized by having excellent detection accuracy, using a primer for amplifying a target in combination with a TaqMan probe in which fluorescence resonance energy transfer (FRET) occurs in the molecule and which specifically binds to the target. The quantitative value of cDNA obtained by applying this TaqMan PCR method is, for example, the number of cycles (Ct value) until cDNA corresponding to each probe and primer reaches the detection standard.

Note that the quantitative value was standardized with a quantitative value of miRNA commonly present in serum samples of healthy individuals and various cancer individuals (hereinafter, “standard miRNA”), considering that the activity level of the entire miRNA may be different for each individual, and the yield of the extraction or amplification reaction may be different for each type of cell or sample in the treatment of the sample. Specifically, correction was performed using a quantitative value of hsa-miR-486-5p (SEQ ID NO: 133) known to have a high expression level in healthy individuals and various cancer individuals. The difference (ΔCt value) by subtracting the Ct value of hsa-miR-486-5p from the Ct value of each miRNA was normalized, so as to be used for correction as an index of the expression level.

The quantitative values of each miRNA standardized as described above are obtained for each individual sample (i.e., for each individual) of healthy individuals, breast cancer patients, pancreatic cancer patients, or colorectal cancer patients. Therefore, if the quantification results are handled for each group of samples of the same type (e.g., a group of samples from healthy individuals), multiple sets of quantitative values are obtained. Such a set of multiple quantitative values is a numerical distribution with some degree of dispersion, since there are individual differences even among a group of samples of the same type of cancer.

In this example, it is assumed that the numerical distribution of the quantitative values of each miRNA observed in a group of similar samples follows a normal distribution. Quantitative values that are outliers for the same normal distribution should be excluded from the analysis, so quantitative values that are outliers were excluded. The average of the quantitative values for each miRNA in each sample group was calculated. The average value can be interpreted as a quantitative value representative of each sample group.

### Results of Quantitating

The average quantitative values of each miRNA in each cancer patient were calculated as described above, and were compared with the average quantitative values of each miRNA in healthy individuals (i.e., converted to the ratio of expression against healthy individuals) and are shown in Table 3.

As shown in Table 3, a total of 94 types of miRNAs were observed. In Table 3, “-” indicates data in which amplification by TaqMan PCR method was not sufficiently observed and the detection reference value was not reached in healthy individuals, breast cancer individuals, pancreatic cancer individuals, and colorectal cancer individuals.

TABLE 3

| | | | Ratio to healthy subject | | |
|---|---|---|---|---|---|
| Sequence | | | Breast | Pancreatic | Colorectal |
| number | Name of miRNA | Base sequence | cancer | cancer | cancer |
| 1 | hsa-miR-128-3p | UCACAGUGAACCGGUCUCUUU | 0.600 | 0.590 | 0.321 |
| 2 | hsa-miR-151a-3p | CUAGACUGAAGCUCCUUGAGG | 0.500 | 0.516 | 0.235 |
| 3 | hsa-miR-143-3p | UGAGAUGAAGCACUGUAGCUC | 0.603 | 0.651 | 0.219 |
| 4 | hsa-miR-4770 | UGAGAUGACACUGUAGCU | 0.551 | 0.603 | 0.203 |
| 5 | hsa-miR-1296-5p | UUAGGGCCCUGGCUCCAUCUCC | 0.452 | 0.413 | 0.173 |
| 6 | hsa-miR-125a-5p | UCCCUGAGACCCUUUAACCUGUGA | 0.551 | 0.564 | 0.200 |
| 7 | hsa-miR-409-3p | GAAUGUUGCUCGGUGAACCCCU | 0.352 | 0.290 | 0.098 |
| 8 | hsa-miR-99a-5p | AACCCGUAGAUCCGAUCUUGUG | 0.690 | 1.040 | 0.344 |
| 9 | hsa-miR-215-5p | AUGACCUAUGAAUUGACAGAC | 0.681 | 1.034 | 0.506 |
| 10 | hsa-miR-126-3p | UCGUACCGUGAGUAAUAAUGCG | 0.467 | 0.456 | 0.204 |
| 11 | hsa-miR-326 | CCUCUGGGCCCUUCCUCCAG | 0.435 | 0.484 | 0.183 |
| 12 | hsa-miR-28-3p | CACUAGAUUGUGAGCUCCUGGA | 0.423 | 0.464 | 0.176 |
| 13 | hsa-miR-425-3p | AUCGGGAAUGUCGUGUCCGCCC | 0.439 | 0.462 | 0.202 |
| 14 | hsa-miR-93-3p | ACUGCUGAGCUAGCACUUCCCG | 0.597 | 0.568 | 0.333 |
| 15 | hsa-miR-24-3p | UGGCUCAGUUCAGCAGGAACAG | 0.417 | 0.443 | 0.183 |
| 16 | hsa-miR-222-3p | AGCUACAUCUGGCUACUGGGU | 0.579 | 0.521 | 0.258 |
| 17 | hsa-miR-361-5p | UUAUCAGAAUCUCCAGGGGUAC | 0.490 | 0.472 | 0.208 |
| 18 | hsa-miR-342-3p | UCUCACACAGAAAUCGCACCCGU | 0.667 | 0.391 | 0.227 |
| 19 | hsa-miR-10b-5p | UACCCUGUAGAACCGAAUUUGUG | 0.869 | 0.723 | 0.352 |
| 20 | hsa-miR-140-3p | UACCACAGGGUAGAACCACGG | 0.829 | 0.782 | 0.595 |
| 21 | hsa-miR-3666 | CAGUGCAAGUGUAGAUGCCGA | — | — | — |
| 22 | hsa-miR-192-5p | CUGACCUAUGAAUUGACAGCC | 0.704 | 1.018 | 0.520 |
| 23 | hsa-miR-4699-5p | AGAAGAUUGCAGAGUAAGUUCC | — | — | — |
| 24 | hsa-miR-3152-5p | AUUGCCUCUGUUCUAACACAAG | — | — | — |
| 25 | hsa-miR-193b-5p | CGGGGUUUUGAGGGCGAGAUGA | — | — | — |
| 26 | hsa-miR-5694 | CAGAUCAUGGGACUGUCUCAG | — | — | — |
| 27 | hsa-miR-3168 | GAGUUCUACAGUCAGAC | 0.576 | 0.554 | 0.300 |
| 28 | hsa-miR-6755-3p | UGUUGUCAUGUUUUUUCCCUAG | — | — | — |
| 29 | hsa-miR-559 | UAAAGUAAAUAUGCACCAAAA | 0.602 | 0.513 | 0.367 |
| 30 | hsa-miR-96-3p | AAUCAUGUGCAGUGCCAAUAUG | — | — | — |
| 31 | hsa-miR-876-3p | UGGUGGUUUACAAAGUAAUUCA | — | — | — |
| 32 | hsa-miR-2052 | UGUUUUGAUAACAGUAAUGU | — | — | — |
| 33 | hsa-miR-514a-5p | UACUCUGGAGAGUGACAAUCAUG | — | — | — |
| 34 | hsa-let-7c-5p | UGAGGUAGUAGGUUGUAUGGUU | 0.504 | 0.499 | 0.289 |
| 35 | hsa-miR-196b-5p | UAGGUAGUUUCCUGUUGUUGGG | 0.498 | 0.530 | 0.224 |
| 36 | hsa-miR-1266-5p | CCUCAGGGCUGUAGAACAGGGCU | — | — | — |
| 37 | hsa-miR-4300 | UGGGAGCUGGACUACUUC | 0.615 | 0.525 | 0.268 |
| 38 | hsa-miR-3151-5p | GGUGGGGCAAUGGGAUCAGGU | 0.342 | 0.429 | 0.224 |
| 39 | hsa-miR-5698 | UGGGGGAGUGCAGUGAUUGUGG | — | — | — |
| 40 | hsa-miR-6756-5p | AGGGUGGGGCUGGAGGUGGGGCU | 0.407 | 0.544 | 0.247 |
| 41 | hsa-miR-615-5p | GGGGGUCCCCGGUGCUCGGAUC | — | — | — |
| 42 | hsa-miR-616-3p | AGUCAUUGGAGGGUUUGAGCAG | — | — | — |
| 43 | hsa-miR-665 | ACCAGGAGGCUGAGGCCCCU | — | — | — |
| 44 | hsa-miR-6765-3p | UCACCUGGCUGGCCCGCCCAG | — | — | — |
| 45 | hsa-miR-6503-3p | GGGACUAGGAUGCAGACCUCC | — | — | — |
| 46 | hsa-miR-1237-5p | CGGGGGCGGGGCCGAAGCGCG | — | — | — |
| 47 | hsa-miR-206 | UGGAAUGUAAGGAAGUGUGUGG | — | — | — |
| 48 | hsa-miR-744-5p | UGCGGGGCUAGGGCUAACAGCA | 0.393 | 0.427 | 0.179 |
| 49 | hsa-miR-6131 | GGCUGGUCAGAUGGGAGUG | — | — | — |
| 50 | hsa-miR-4732-5p | UGUAGAGCAGGGAGCAGGAAGCU | 1.065 | 0.909 | 1.148 |
| 51 | hsa-miR-542-3p | UGUGACAGAUUGAUAACUGAAA | 0.676 | 0.543 | 0.304 |
| 52 | hsa-miR-127-3p | UCGGAUCCGUCUGAGCUUGGCU | 0.305 | 0.292 | 0.096 |
| 53 | hsa-miR-1246 | AAUGGAUUUUUGGAGCAGG | 0.218 | 0.353 | 0.146 |
| 54 | hsa-miR-485-3p | GUCAUACACGGCUCUCCUCUCU | 0.334 | 0.223 | 0.078 |
| 55 | hsa-miR-485-5p | AGAGGCUGGCCGUGAUGAAUUC | 0.772 | 1.038 | 0.855 |
| 56 | hsa-miR-5585-3p | CUGAAUAGCUGGGACUACAGGU | — | — | — |
| 57 | hsa-miR-4466 | GGGUGCGGGCCGGCGGGG | 0.412 | 0.459 | 0.237 |
| 58 | hsa-miR-19b-3p | UGUGCAAAUCCAUGCAAAACUGA | 0.873 | 0.802 | 0.701 |
| 59 | hsa-miR-424-3p | CAAAACGUGAGGCGCUGCUAU | 0.673 | 0.686 | 0.356 |
| 60 | hsa-miR-324-5p | CGCAUCCCCUAGGGCAUUGGUG | 0.525 | 0.573 | 0.305 |
| 61 | hsa-miR-4433b-5p | AUGUCCCACCCCCACUCCUGU | 0.488 | 0.412 | 0.196 |
| 62 | hsa-miR-150-5p | UCUCCCAACCCUUGUACCAGUG | 0.660 | 0.330 | 0.226 |
| 63 | hsa-miR-146a-5p | UGAGAACUGAAUUCCAUGGGUU | 0.186 | 0.393 | 0.156 |
| 64 | hsa-miR-483-3p | UCACUCCUCUCCUCCCGUCUU | 0.900 | 1.318 | 0.440 |
| 65 | hsa-miR-27b-5p | AGAGCUUAGCUGAUUGGUGAAC | — | — | — |
| 66 | hsa-miR-493-3p | UGAAGGUCUACUGUGUGCCAGG | — | — | — |
| 67 | hsa-miR-3150b-3p | UGAGGAGAUCGUCGAGGUUGG | — | — | — |
| 68 | hsa-miR-652-3p | AAUGGCGCCACUAGGGUUGUG | 0.449 | 0.511 | 0.240 |
| 69 | hsa-miR-9-5p | UCUUUGGUUAUCUAGCUGUAUGA | 0.412 | 0.442 | 0.190 |
| 70 | hsa-miR-3182 | GCUUCUGUAGUGUAGUC | 0.511 | 0.580 | 0.324 |
| 71 | hsa-miR-4765 | UGAGUGAUUGAUAGCUAUGUUC | — | — | — |
| 72 | hsa-miR-758-3p | UUUGUGACCUGGUCCACUAACC | — | — | — |
| 73 | hsa-miR-1229-3p | CUCUCACCACUGCCCUCCCACAG | — | — | — |
| 74 | hsa-miR-15a-5p | UAGCAGCACAUAAUGGUUUGUG | 0.693 | 0.679 | 0.535 |
| 75 | hsa-miR-21-5p | UAGCUUAUCAGACUGAUGUUGA | 0.472 | 0.519 | 0.245 |
| 76 | hsa-miR-767-5p | UGCACCAUGGUUGUCUGAGCAUG | — | — | — |
| 77 | hsa-miR-6797-3p | UGCAUGACCCUUCCCUCCCCAC | — | — | — |
| 78 | hsa-miR-32-5p | UAUUGCACAUUACUAAGUUGCA | 0.591 | 0.415 | 0.301 |
| 79 | hsa-miR-6830-3p | UGUCUUUCUUCUCUCCCUUGCAG | — | — | — |
| 80 | hsa-miR-576-3p | AAGAUGUGGAAAAAUUGGAAUC | 0.660 | 0.736 | 0.402 |
| 81 | hsa-miR-6873-3p | UUCUCUCUGUCUUUCUCUCUCAG | — | — | — |
| 82 | hsa-miR-1249-3p | ACGCCCUUCCCCCCCUUCUUCA | 0.612 | 0.543 | 0.243 |
| 83 | hsa-miR-433-3p | AUCAUGAUGGGCUCCUCGGUGU | 0.260 | 0.303 | 0.092 |
| 84 | hsa-miR-6886-3p | UGCCCUUCUCUCCUCCUGCCU | 0.387 | 0.349 | 0.224 |
| 85 | hsa-miR-329-5p | GAGGUUUUCUGGGUUUCUGUUUC | 0.394 | 0.392 | 0.247 |
| 86 | hsa-miR-6859-5p | GAGAGGAACAUGGGCUCAGGACA | 0.630 | 0.510 | 0.470 |
| 87 | hsa-miR-15b-5p | UAGCAGCACAUCAUGGUUUACA | 0.425 | 0.429 | 0.234 |
| 88 | hsa-miR-25-3p | CAUUGCACUUGUCUCGGUCUGA | 0.836 | 0.822 | 0.681 |
| 89 | hsa-miR-451a | AAACCGUUACCAUUACUGAGUU | 1.068 | 1.055 | 1.042 |
| 90 | hsa-miR-425-5p | AAUGACACGAUCACUCCCGUUGA | 0.633 | 0.579 | 0.389 |
| 91 | hsa-miR-93-5p | CAAAGUGCUGUUCGUGCAGGUAG | 0.763 | 0.668 | 0.510 |
| 92 | hsa-miR-148a-3p | UCAGUGCACUACAGAACUUUGU | 0.538 | 0.642 | 0.299 |
| 93 | hsa-miR-103a-3p | AGCAGCAUUGUACAGGGCUAUGA | 0.475 | 0.456 | 0.285 |
| 94 | hsa-miR-3615 | UCUCUCGGCUCCUCGCGGCUC | 0.595 | 0.718 | 0.437 |
| 95 | hsa-miR-221-3p | AGCUACAUUGUCUGCUGGGUUUC | 0.416 | 0.470 | 0.201 |
| 96 | hsa-miR-484 | UCAGGCUCAGUCCCCUCCCGAU | 0.597 | 0.610 | 0.390 |
| 97 | hsa-miR-382-5p | GAAGUUGUUCGUGGUGGAUUCG | 0.257 | 0.264 | 0.090 |
| 98 | hsa-miR-101-3p | UACAGUACUGUGAUAACUGAA | 0.970 | 0.838 | 0.809 |
| 99 | hsa-miR-22-3p | AAGCUGCCAGUUGAAGAACUGU | 0.474 | 0.537 | 0.354 |
| 100 | hsa-miR-27b-3p | UUCACAGUGGCUAAGUUCUGC | 0.506 | 0.534 | 0.222 |
| 101 | hsa-miR-155-5p | UUAAUGCUAAUCGUGAUAGGGGUU | 0.475 | 0.434 | 0.235 |
| 102 | hsa-miR-196a-5p | UAGGUAGUUUCAUGUUGUUGGG | 0.456 | 0.435 | 0.261 |
| 103 | hsa-miR-22-5p | AGUUCUUCAGUGGCAAGCUUUA | 0.555 | 0.585 | 0.332 |
| 104 | hsa-miR-574-5p | UGAGUGUGUGUGUGUGAGUGUGU | 0.437 | 0.415 | 0.279 |
| 105 | hsa-miR-584-5p | UUAUGGUUUGCCUGGGACUGAG | 0.388 | 0.439 | 0.199 |
| 106 | hsa-miR-99b-5p | CACCCGUAGAACCGACCUUGCG | 0.380 | 0.377 | 0.162 |
| 107 | hsa-miR-664a-3p | UAUUCAUUUAUCCCCAGCCUACA | 0.509 | 0.413 | 0.195 |
| 108 | hsa-miR-18a-5p | UAAGGUGCAUCUAGUGCAGAUAG | 0.541 | 0.547 | 0.346 |
| 109 | hsa-miR-181b-5p | AACAUUCAUUGCUGUCGGUGGGU | 0.449 | 0.440 | 0.213 |
| 110 | hsa-miR-1-3p | UGGAAUGUAAAGAAGUAUGUAU | 0.511 | 0.575 | 0.266 |
| 111 | hsa-miR-660-5p | UACCCAUUGCAUAUCGGAGUUG | 0.828 | 0.686 | 0.553 |
| 112 | hsa-miR-27a-3p | UUCACAGUGGCUAAGUUCCGC | 0.472 | 0.455 | 0.203 |
| 113 | hsa-miR-1276 | UAAAGAGCCCUGUGGAGACA | — | — | — |
| 114 | hsa-miR-769-5p | UGAGACCUCUGGGUUCUGAGCU | 0.499 | 0.521 | 0.235 |
| 115 | hsa-miR-148b-3p | UCAGUGCAUCACAGAACUUUGU | 0.544 | 0.508 | 0.313 |
| 116 | hsa-miR-5590-3p | AAUAAAGUUCAUGUAUGGCAA | — | — | — |
| 117 | hsa-miR-3927-5p | GCCUAUCACAUAUCUGCCUGU | — | — | — |
| 118 | hsa-miR-1910-5p | CCAGUCCUGUGCCUGCCGCCU | — | — | — |
| 119 | hsa-miR-509-3-5p | UACUGCAGACGUGGCAAUCAUG | — | — | — |
| 120 | hsa-miR-338-5p | AACAAUAUCCUGGUGCUGAGUG | 0.439 | 0.445 | 0.160 |
| 121 | hsa-miR-513-5p | UUCACAGGGAGGUGUCAU | — | — | — |
| 122 | hsa-miR-520e-3p | AAAGUGCUUCCUUUUUGAGGG | 0.715 | 0.576 | 0.428 |
| 123 | hsa-miR-4657 | AAUGUGGAAGUGGUCUGAGGCAU | — | — | — |
| 124 | hsa-miR-3155a | CCAGGCUCUGCAGUGGGAACU | 0.431 | 0.421 | 0.249 |
| 125 | hsa-miR-132-3p | UAACAGUCUACAGCCAUGGUCG | 0.271 | 0.296 | 0.138 |
| 126 | hsa-miR-6872-5p | UCUCGCAUCAGGAGGCAAGG | — | — | — |
| 127 | hsa-miR-16-5p | UAGCAGCACGUAAAUAUUGGCG | 1.055 | 0.928 | 0.916 |
| 128 | hsa-let-7a-5p | UGAGGUAGUAGGUUGUAUAGUU | 0.359 | 0.355 | 0.181 |
| 129 | hsa-let-7i-5p | UGAGGUAGUAGUUUGUGCUGUU | 0.518 | 0.536 | 0.332 |
| 130 | hsa-miR-30d-5p | UGUAAACAUCCCCGACUGGAAG | 0.700 | 0.672 | 0.409 |
| 131 | hsa-let-7f-5p | UGAGGUAGUAGAUUGUAUAGUU | 0.335 | 0.309 | 0.146 |
| 132 | hsa-miR-3074-5p | GUUCCUGCUGAACUGAGCCAG | — | — | — |

### Marker Selection

Among 94 types of the miRNAs, miRNAs characteristically present in breast cancer, pancreatic cancer or colorectal cancer, that is, markers of each cancer type were selected.

Whether or not the miRNA is characteristic for each cancer type was determined based on whether or not the ratio between log₂fold value of the quantitative value (ex. average), which represents for a group of sample of a certain cancer type (hereinafter, “sample group A”), and log₂fold value of the quantitative value (ex. average), which represents for a group of sample of the other cancer type or healthy individuals (hereinafter, “sample group B”), is different by twice or more. In other words, when a certain miRNA has a quantitative value confirmed in the sample group A that is twice or more or ½ or less of a quantitative value confirmed in the sample group B, the miRNA was determined to be characteristic for the sample group A and to be a marker indicating the morbidity of the cancer type classified to the sample group A.

However, as mentioned above, the multiple quantitative values for each miRNA in each sample group are numerically distributed with some degree of variance. Depending on the degree of overlap in the numerical distribution, it may not be possible to distinguish between Sample Group A and Sample Group B. Therefore, by verifying whether or not sample group A can be statistically distinguished from sample group B, it is possible to select marker candidates with higher accuracy.

In this example, as a candidate for a marker for pancreatic cancer, miRNAs were selected whose average quantitative value in the sample group of pancreatic cancer patients was more than twice or less than half of the average quantitative value in the sample group of healthy individuals, breast cancer patients, or colorectal cancer patients. In addition, it was tested whether the selected pancreatic cancer markers were statistically significant or not.

Similarly, as a candidate for a marker for colorectal cancer, miRNAs were selected whose average quantitative value in the sample group of colorectal cancer patients was more than twice or less than half of the average quantitative value in the sample group of healthy individuals, breast cancer patients, or pancreatic cancer patients, and tested whether the selected colorectal cancer markers were statistically significant or not.

### Results of Marker Selection

The miRNAs selected as markers are shown in Table 4.

TABLE 4

| Sequence | Marker | |
|---|---|---|
| number | (Name of miRNA) | Base sequence |
| 1 | hsa-miR-128-3p | ucacagugaaccggucucuuu |
| 2 | hsa-miR-151a-3p | cuagacugaagcuccuugagg |
| 3 | hsa-miR-143-3p | ugagaugaagcacuguagcuc |
| 4 | hsa-miR-4770 | ugagaugacacuguagcu |
| 5 | hsa-miR-1296-5p | uuagggcccuggcuccaucucc |
| 6 | hsa-miR-125a-5p | ucccugagacccuuuaaccuguga |
| 7 | hsa-miR-409-3p | gaauguugcucggugaaccccu |
| 8 | hsa-miR-99a-5p | aacccguagauccgaucuugug |
| 9 | hsa-miR-215-5p | augaccuaugaauugacagac |
| 10 | hsa-miR-126-3p | ucguaccgugaguaauaaugcg |
| 11 | hsa-miR-326 | ccucugggcccuuccuccag |
| 12 | hsa-miR-28-3p | cacuagauugugagcuccugga |
| 13 | hsa-miR-425-3p | aucgggaaugucguguccgccc |
| 14 | hsa-miR-93-3p | acugcugagcuagcacuucccg |
| 15 | hsa-miR-24-3p | uggcucaguucagcaggaacag |
| 16 | hsa-miR-222-3p | agcuacaucuggcuacugggu |
| 17 | hsa-miR-361-5p | uuaucagaaucuccagggguac |
| 18 | hsa-miR-342-3p | ucucacacagaaaucgcacccgu |
| 19 | hsa-miR-10b-5p | uacccuguagaaccgaauuugug |

As shown in Table 4, a total of 19 types of cancer markers of each cancer type could be identified. For example, hsa-miR-128-3p (SEQ ID NO:1), hsa-miR-151a-3p (SEQ ID NO:2), hsa-miR-143-3p (SEQ ID NO:3), hsa-miR-4770 (SEQ ID NO:4), hsa-miR-1296-5p (SEQ ID NO:5), hsa-miR-125a-5p (SEQ ID NO:6), hsa-miR-409-3p (SEQ ID NO:7), hsa-miR-99a-5p (SEQ ID NO:8), hsa-miR-215-5p (SEQ ID NO:9), hsa-miR-126-3p (SEQ ID NO:10), hsa-miR-326 (SEQ ID NO:11), hsa-miR-28-3p (SEQ ID NO:12), hsa-miR-425-3p (SEQ ID NO:13), hsa-miR-93-3p (SEQ ID NO:14), hsa-miR-24-3p (SEQ ID NO:15), hsa-miR-222-3p (SEQ ID NO:16), hsa-miR-361-5p (SEQ ID NO:17), hsa-miR-342-3p (SEQ ID NO:18) and hsa-miR-10b-5p (SEQ ID NO:19) has been found to be a marker of a low expression target miRNA indicating that a subject has breast cancer, pancreatic cancer or colorectal cancer, because its abundance in the subject is less of its abundance in healthy individuals.

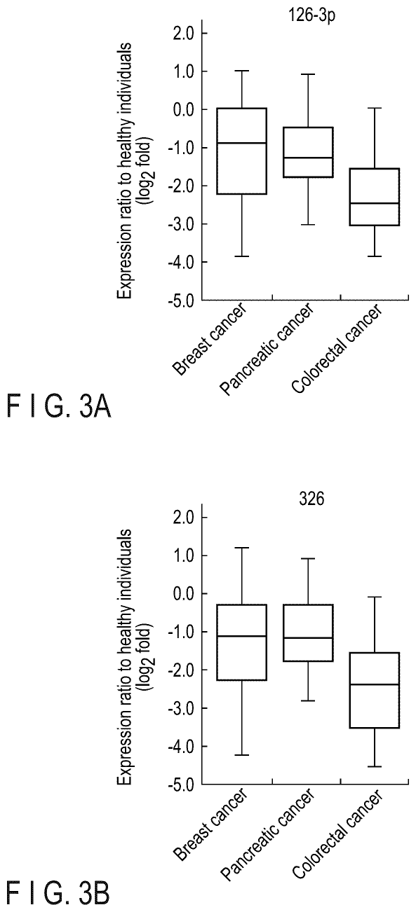

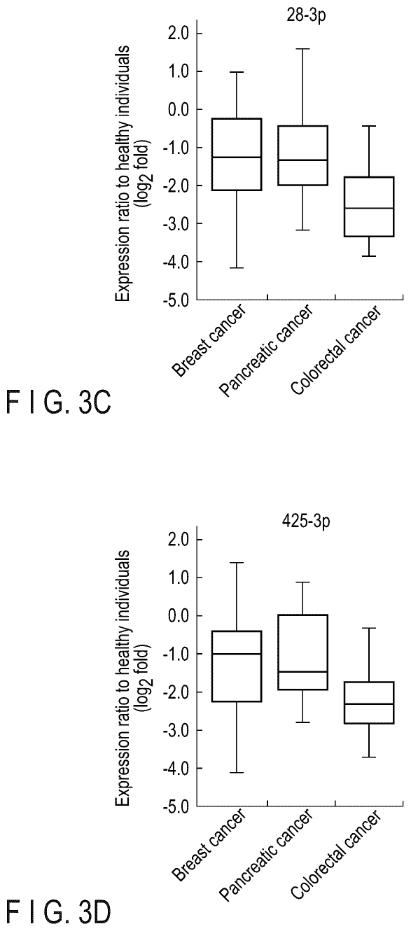

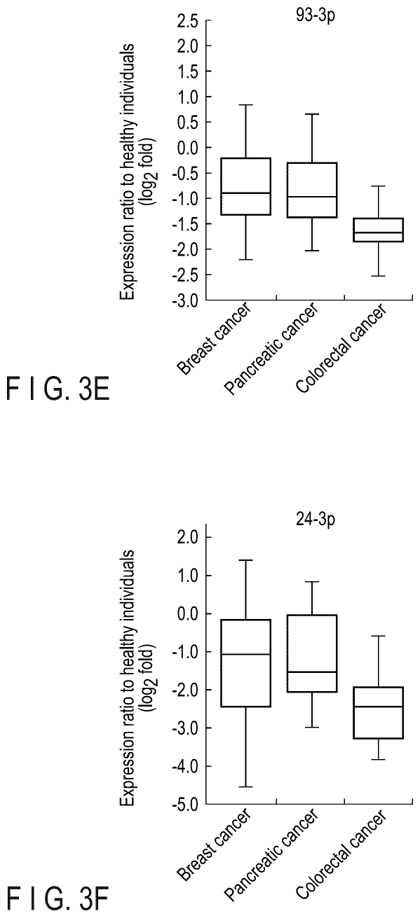

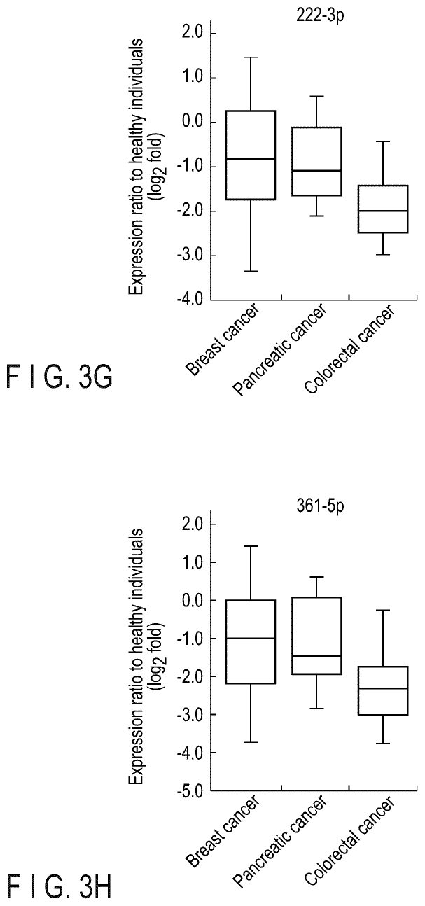

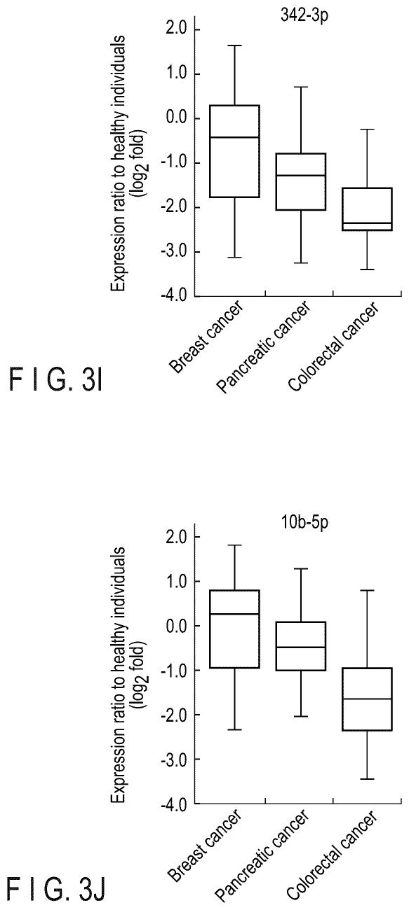

The ratio of each miRNA of SEQ ID NO: 10 to 19 to the healthy individuals is shown in FIGS. 3A to 3J, respectively (the box diagrams of SEQ ID NO: 1 to 9 are omitted because the same results are shown in Example 2 below). FIGS. 3A to 3J are box plots, where the horizontal axis indicates each cancer type that each sample is affected with and the vertical axis indicates the ratio of each miRNA measured in each sample to healthy individuals' expression (log₂fold value).

Here, it was tested whether each miRNA marker showing the distribution of quantitative values shown in FIG. 3A to FIG. 3J could be discriminated from healthy individuals or other cancer types. Specifically, for example, the control was a healthy individual and the subject was a breast, pancreatic, or colorectal cancer patient, and it was tested whether the range of possible quantitative values of each miRNA was significantly different between the two groups. Similarly, it was also tested whether each marker was statistically significantly discriminative when the control was a colon cancer patient and the subject was a breast or pancreatic cancer patient, or when the control was a breast cancer patient and the subject was a pancreatic cancer patient, and so on.

The results of statistical analysis are shown in Tables 5 and 6. In Table 5, “*” indicates significance when the level of significance (p-value) is set to a value smaller than 0.05, “**” indicates significance when the level of significance (p-value) is set to a value smaller than 0.01, “***” indicates significance when the level of significance (p-value) is set to a value smaller than 0.001, and “n.s” indicates no significant difference. In Table 6, “decrease” indicates that the quantitative value of the subject is smaller than that of the control, “increase” indicates that the quantitative value of the subject is larger than that of the control, and “n.s” indicates that it is not a significant difference. Each miRNA is indicated by an abbreviation (for example, the hsa-miR-126-3p is described as “126-3p”).

TABLE 5

| Control | Subject | 126-3p | 326 | 28-3p | 425-3p | 93-3p | 24-3p | 222-3p | 361-5p | 342-3p | 10b-5p |
|---|---|---|---|---|---|---|---|---|---|---|---|
| Non- | Breast | *** | *** | *** | *** | ** | *** | * | ** | n.s. | n.s. |
| Cancer | cancer | | | | | | | | | | |
| | Pancreatic | *** | ** | ** | ** | *** | ** | ** | ** | *** | n.s. |
| | cancer | | | | | | | | | | |
| | Colorectal | *** | *** | *** | *** | *** | *** | *** | *** | *** | *** |
| | cancer | | | | | | | | | | |
| Breast | Pancreatic | n.s. | n.s. | n.s. | n.s. | n.s. | n.s. | n.s. | n.s. | * | n.s. |
| cancer | cancer | | | | | | | | | | |
| | Colorectal | *** | *** | *** | ** | *** | ** | *** | *** | *** | *** |
| | cancer | | | | | | | | | | |
| Pancreatic | Colorectal | *** | *** | *** | *** | ** | *** | *** | ** | * | ** |
| cancer | cancer | | | | | | | | | | |

* p < 0.05,
** p < 0.01,
*** p < 0.001,
n.s; No significant difference

TABLE 6

| Control | Subject | 126-3p | 326 | 28-3p | 425-3p | 93-3p | 24-3p | 222-3p | 361-5p | 342-3p | 10b-5p |
|---|---|---|---|---|---|---|---|---|---|---|---|
| Non- | Breast | Decrease | Decrease | Decrease | Decrease | Decrease | Decrease | Decrease | Decrease | n.s. | n.s. |
| Cancer | cancer | | | | | | | | | | |
| | Pancreatic | Decrease | Decrease | Decrease | Decrease | Decrease | Decrease | Decrease | Decrease | Decrease | n.s. |
| | cancer | | | | | | | | | | |
| | Colorectal | Decrease | Decrease | Decrease | Decrease | Decrease | Decrease | Decrease | Decrease | Decrease | Decrease |
| | cancer | | | | | | | | | | |
| Breast | Pancreatic | n.s. | n.s. | n.s. | n.s. | n.s. | n.s. | n.s. | n.s. | Decrease | n.s. |
| cancer | cancer | | | | | | | | | | |
| | Colorectal | Decrease | Decrease | Decrease | Decrease | Decrease | Decrease | Decrease | Decrease | Decrease | Decrease |
| | cancer | | | | | | | | | | |
| Pancreatic | Colorectal | Decrease | Decrease | Decrease | Decrease | Decrease | Decrease | Decrease | Decrease | Decrease | Decrease |
| cancer | cancer | | | | | | | | | | |

* p < 0.05,
** p < 0.01,
*** p < 0.001,
n.s; No significant difference

As shown in Tables 5 and 6, it can be seen that by measuring and comparing the abundance of each miRNA of SEQ ID NO: 10 to 19, it is possible to distinguish statistically significantly between controls and subjects. Thus, each miRNA of SEQ ID NO:10 to 19 was shown to be a marker for breast, pancreatic, and colorectal cancers, all of which can be determined as likely to be affected in the subject.

### Example 2: More Extensive Marker Search and Selection

A search for miRNA markers using the similar method as in Example 1 was performed with an increased number and variety of samples.

### Preparation of Samples

As samples, breast cancer patient sera, pancreatic cancer patient sera, lung cancer patient serum, gastric cancer patient serum, colorectal cancer patient sera, and healthy individual sera were prepared for a total of 576 samples. The number of samples for each was 100 from breast cancer patients, 99 from pancreatic cancer patients, 99 from lung cancer patients, 99 from gastric cancer patients, 98 from colon cancers patients, and 81 from healthy individuals, as shown in Table 4.

Treatment of Samples and Quantification of miRNAs

The samples were subjected to the similar method as in Example 1 to extract miRNAs, and various miRNAs were quantified. Each quantitative value was standardized with the quantitative value of hsa-miR-486-5p, a standard miRNA, and the expression ratios of various miRNAs among the groups of healthy individuals, breast cancer, lung cancer, pancreatic cancer, gastric cancer, and colorectal cancer were determined.

### Quantitative Results and Statistical Validation

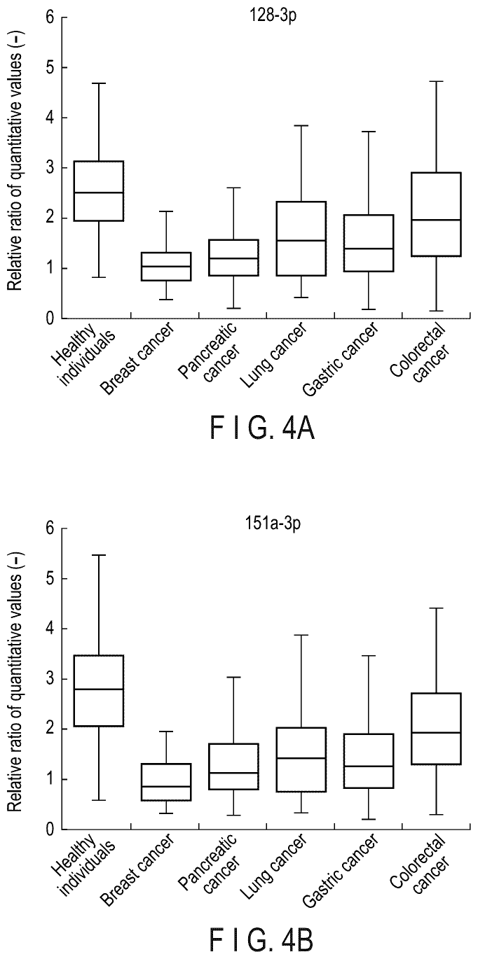

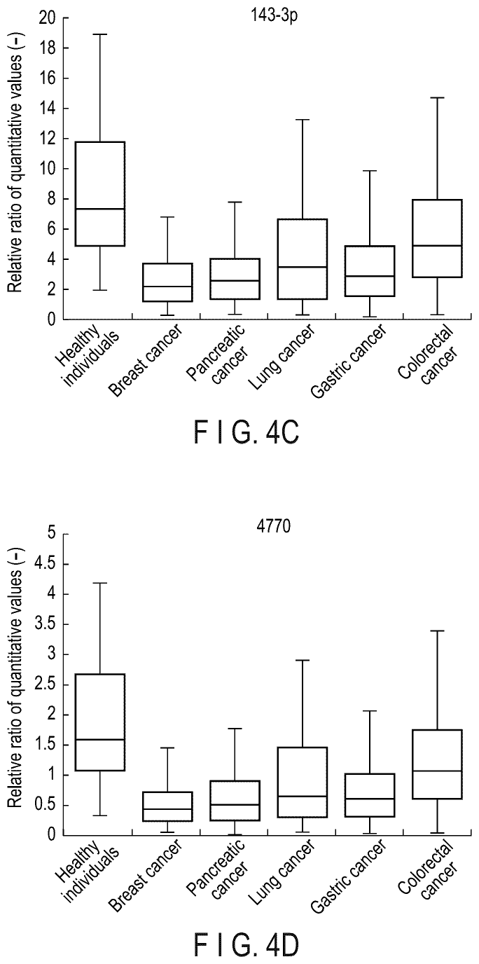

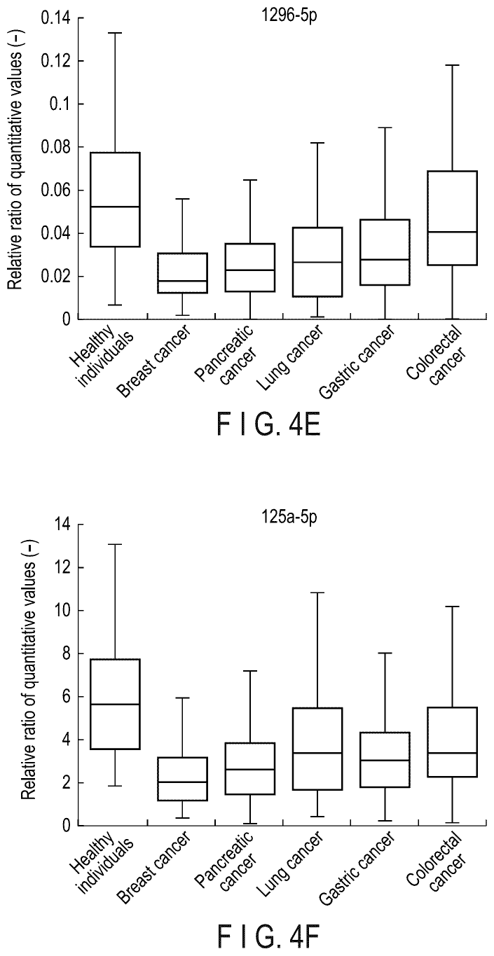

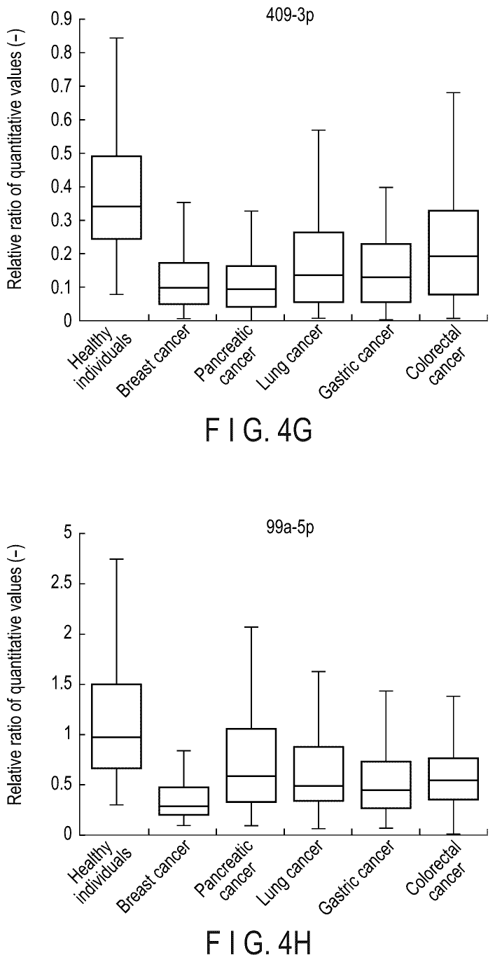

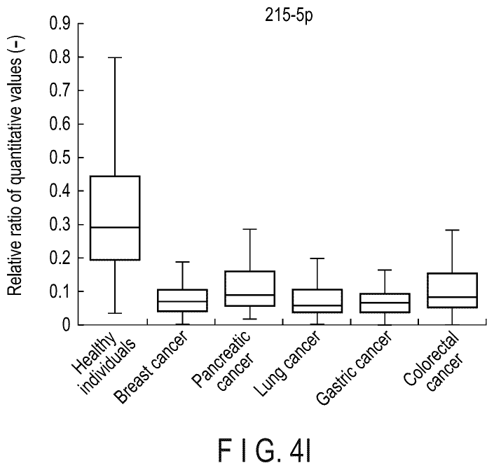

The quantitative values of the 9 types of miRNA markers, which is identified in Example 2, are shown in FIGS. 4A to 4I. FIGS. 4A to 4I are box plots, where the horizontal axis indicates each cancer type that each sample is affected with and the vertical axis indicates the relative ratio of the quantitative values measured in each sample.

Each miRNA marker showing the distribution of quantitative values shown in FIG. 4A to FIG. 4I was tested to see if it could be discriminated from healthy individuals or other cancer types. Specifically, for example, the control was a healthy individual and the subject was a patient with breast, pancreatic, lung, stomach, or colorectal cancer, and whether the range of possible quantitative values of each miRNA was significantly different between the two groups was verified. Similarly, it was tested whether each marker was statistically significantly discriminative when the control was colorectal cancer patients and the target was breast, pancreatic, lung, or gastric cancer patients; when the control was breast cancer patients and the target was pancreatic, lung, or gastric cancer patients; and when the control was lung cancer patients and the target was pancreatic or gastric cancer patients.

The results of statistical analysis are shown in Tables 7 and 8. In Tables 7, “*” indicates significance when the level of significance (p-value) is set to a value smaller than 0.05, “**” indicates significance when the level of significance (p-value) is set to a value smaller than 0.01, “***” indicates significance when the level of significance (p-value) is set to a value smaller than 0.001, and “n.s” indicates no significant difference. In Table 8, “decrease” indicates that the quantitative value of the subject is smaller than that of the control, “increase” indicates that the quantitative value of the subject is larger than that of the control, and “n.s” indicates that it is not a significant difference. Each miRNA is indicated by an abbreviation (for example, the hsa-miR-190-5p is described as “190-5p”).

TABLE 7

| Control | Subject | 128-3p | 151a-3p | 143-3p | 4770 | 1296-5p | 125a-5p | 409-3p | 99a-5p | 215-5p |
|---|---|---|---|---|---|---|---|---|---|---|
| Non- | Breast | *** | *** | *** | *** | *** | *** | *** | *** | *** |
| Cancer | cancer | | | | | | | | | |
| | Pancreatic | *** | *** | *** | *** | *** | *** | *** | * | *** |
| | cancer | | | | | | | | | |
| | Lung | *** | *** | *** | *** | *** | *** | *** | ** | *** |
| | cancer | | | | | | | | | |
| | Gastric | *** | *** | *** | *** | *** | *** | *** | n.s. | *** |
| | cancer | | | | | | | | | |
| | Colorectal | ** | *** | n.s. | ** | n.s. | ** | ** | * | *** |
| | cancer | | | | | | | | | |
| Colorectal | Breast | *** | *** | *** | *** | *** | ** | ** | n.s. | n.s. |
| cancer | cancer | | | | | | | | | |
| | Pancreatic | *** | *** | *** | *** | *** | n.s. | ** | n.s. | n.s. |
| | cancer | | | | | | | | | |
| | Lung | ** | * | * | n.s. | *** | n.s. | n.s. | n.s. | n.s. |
| | cancer | | | | | | | | | |
| | Gastric | *** | *** | ** | ** | *** | n.s. | n.s. | n.s. | n.s. |
| | cancer | | | | | | | | | |
| Lung | Breast | *** | ** | * | * | n.s. | ** | n.s. | n.s. | n.s. |
| cancer | cancer | | | | | | | | | |
| | Pancreatic | n.s. | n.s. | n.s. | n.s. | n.s. | n.s. | n.s. | n.s. | n.s. |
| | cancer | | | | | | | | | |
| | Gastric | n.s. | n.s. | n.s. | n.s. | n.s. | n.s. | n.s. | n.s. | n.s. |
| | cancer | | | | | | | | | |
| Gastric | Breast | * | n.s. | n.s. | n.s. | n.s. | ** | n.s. | * | n.s. |
| cancer | cancer | | | | | | | | | |
| | Pancreatic | n.s. | n.s. | n.s. | n.s. | n.s. | n.s. | n.s. | n.s. | n.s. |
| | cancer | | | | | | | | | |
| Pancreatic | Breast | n.s. | n.s. | n.s. | n.s. | n.s. | n.s. | n.s. | n.s. | n.s. |
| cancer | cancer | | | | | | | | | |

* p < 0.05,
** p < 0.01,
*** p < 0.001,
n.s; No significant difference

TABLE 8

| Control | Subject | 128-3p | 151a-3p | 143-3p | 4770 | 1296-5p |
|---|---|---|---|---|---|---|
| Non- | Breast | Decrease | Decrease | Decrease | Decrease | Decrease |
| Cancer | cancer | | | | | |
| | Pancreatic | Decrease | Decrease | Decrease | Decrease | Decrease |
| | cancer | | | | | |
| | Lung | Decrease | Decrease | Decrease | Decrease | Decrease |
| | cancer | | | | | |
| | Gastric | Decrease | Decrease | Decrease | Decrease | Decrease |
| | cancer | | | | | |
| | Colorectal | Decrease | Decrease | n.s. | Decrease | n.s. |
| | cancer | | | | | |
| Colorectal | Breast | Decrease | Decrease | Decrease | Decrease | Decrease |
| cancer | cancer | | | | | |
| | Pancreatic | Decrease | Decrease | Decrease | Decrease | Decrease |
| | cancer | | | | | |
| | Lung | Decrease | Decrease | Decrease | n.s. | Decrease |
| | cancer | | | | | |
| | Gastric | Decrease | Decrease | Decrease | Decrease | Decrease |
| | cancer | | | | | |
| Lung | Breast | Decrease | Decrease | Decrease | Decrease | n.s. |
| cancer | cancer | | | | | |
| | Gastric | n.s. | n.s. | n.s. | n.s. | n.s. |
| | cancer | | | | | |
| | Pancreatic | n.s. | n.s. | n.s. | n.s. | n.s. |
| | cancer | | | | | |
| Gastric | Breast | Decrease | n.s. | n.s. | n.s. | n.s. |
| cancer | cancer | | | | | |
| | Pancreatic | n.s. | n.s. | n.s. | n.s. | n.s. |
| | cancer | | | | | |
| Pancreatic | Breast | n.s. | n.s. | n.s. | n.s. | n.s. |
| cancer | cancer | | | | | |

| Control | Subject | 125a-5p | 409-3p | 99a-5p | 215-5p |
|---|---|---|---|---|---|
| Non- | Breast | Decrease | Decrease | Decrease | Decrease |
| Cancer | cancer | | | | |
| | Pancreatic | Decrease | Decrease | Decrease | Decrease |
| | cancer | | | | |
| | Lung | Decrease | Decrease | Decrease | Decrease |
| | cancer | | | | |
| | Gastric | Decrease | Decrease | n.s. | Decrease |
| | cancer | | | | |
| | Colorectal | Decrease | Decrease | Decrease | Decrease |
| | cancer | | | | |
| Colorectal | Breast | Decrease | Decrease | n.s. | n.s. |
| cancer | cancer | | | | |
| | Pancreatic | n.s. | Decrease | n.s. | n.s. |
| | cancer | | | | |
| | Lung | n.s. | n.s. | n.s. | n.s. |
| | cancer | | | | |
| | Gastric | n.s. | n.s. | n.s. | n.s. |
| | cancer | | | | |
| Lung | Breast | Decrease | n.s. | n.s. | n.s. |
| cancer | cancer | | | | |
| | Gastric | n.s. | n.s. | n.s. | n.s. |
| | cancer | | | | |
| | Pancreatic | n.s. | n.s. | n.s. | n.s. |
| | cancer | | | | |
| Gastric | Breast | Decrease | n.s. | Decrease | n.s. |
| cancer | cancer | | | | |
| | Pancreatic | n.s. | n.s . | n.s. | n.s. |
| | cancer | | | | |
| Pancreatic | Breast | n.s. | n.s. | n.s. | n.s. |
| cancer | cancer | | | | |

* p < 0.05,
** p < 0.01,
*** p < 0.001,
n.s; No significant difference

As described above, it can be seen that by measuring and comparing the abundance of each miRNA of SEQ ID NO: 1 to 9, it is possible to distinguish statistically significantly between controls and subjects. Thus, each miRNA of SEQ ID NO: 1 to 9 was shown to be a marker for determining the probability of having breast, pancreatic, lung, gastric, and colorectal cancer in the subject.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An analytical method including:
quantifying a miRNA selected from the group consisting of hsa-miR-128-3p, hsa-miR-151a-3p, hsa-miR-143-3p, hsa-miR-4770, hsa-miR-1296-5p, hsa-miR-125a-5p, hsa-miR-409-3p, hsa-miR-99a-5p and hsa-miR-215-5p in a subject, and
determining a probability that the subject has breast cancer, pancreatic cancer, lung cancer, gastric cancer, or colorectal cancer.

2. The method of claim 1, further comprising quantifying the miRNA in a control,
wherein the determination is made by comparing a quantitative value of the miRNA in the subject with a quantitative value of the miRNA in the control to determine a probability that the subject has breast cancer, pancreatic cancer, lung cancer, gastric cancer or colorectal cancer.

3. The method of claim 2, wherein the control is a healthy individual or an individual known to have at least one of breast, pancreatic, lung, gastric and colorectal cancer.

4. The method of claim 3, wherein the control is a healthy individual, and wherein the determination includes determining that the subject has breast, pancreatic, lung, gastric, or colorectal cancer when a quantitative value of hsa-miR-128-3p, hsa-miR-151a-3p, hsa-miR-4770, hsa-miR-125a-5p, hsa-miR-409-3p or hsa-miR-215-5p in the subject is less than a quantitative value in the control.

5. The method of claim 3, wherein the control is a healthy individual, and wherein the determination includes determining that the subject has breast, pancreatic, lung, or gastric cancer when a quantitative value of hsa-miR-143-3p or hsa-miR-1296-5p in the subject is less than a quantitative value in the control.

6. The method of claim 3, wherein the control is a healthy individual, and wherein the determination includes determining that the subject has breast, pancreatic, lung, or colorectal cancer when a quantitative value of hsa-miR-99a-5p in the subject is less than a quantitative value in the control.

7. The method of claim 3, wherein the control is a colorectal cancer, and wherein the determination includes determining that the subject has breast, pancreatic, lung, or gastric cancer when a quantitative value of hsa-miR-128-3p, hsa-miR-151a-3p, hsa-miR-143-3p or hsa-miR-1296-5p in the subject is less than a quantitative value in the control.

8. The method of claim 3, wherein the control is a colorectal cancer, and wherein the determination includes determining that the subject has breast, pancreatic or gastric cancer when a quantitative value of hsa-miR-4770 in the subject is less than a quantitative value in the control.

9. The method of claim 3, wherein the control is a colorectal cancer, and wherein the determination includes determining that the subject has breast or pancreatic cancer when a quantitative value of hsa-miR-409-3p in the subject is less than a quantitative value in the control.

10. An analytical method including:
quantifying a miRNA selected from the group consisting of hsa-miR-128-3p, hsa-miR-151a-3p, hsa-miR-143-3p, hsa-miR-4770, hsa-miR-125a-5p and hsa-miR-99a-5p in a subject, and
determining a probability that the subject has breast cancer.

11. The method of claim 10, further comprising quantifying the miRNA in a control, wherein the determination is made by comparing a quantitative value of the miRNA in the subject with a quantitative value of the miRNA in the control to determine a probability that the subject has breast cancer.

12. The method of claim 11, wherein the control is an individual known to have at least one of pancreatic cancer, lung cancer, gastric cancer, or colorectal cancer.

13. The method of claim 11, wherein the control has a lung or gastric cancer, and wherein the determination includes determining that the subject has breast cancer when a quantitative value of hsa-miR-128-3p in the subject is less than a quantitative value in the control.

14. The method of claim 11, wherein the control has a lung cancer, and wherein the determination includes determining that the subject has breast cancer when a quantitative value of hsa-miR-151a-3p, hsa-miR-143-3p or hsa-miR-4770 in the subject is less than a quantitative value in the control.

15. The method of claim 11, wherein the control has a colorectal, lung or gastric cancer, and wherein the determination includes determining that the subject has breast cancer when a quantitative value of hsa-miR-125a-5p in the subject is less than a quantitative value in the control.

16. The method of claim 11, wherein the control has a gastric cancer, and wherein the determination includes determining that the subject has breast cancer when a quantitative value of hsa-miR-99a-5p in the subject is less than a quantitative value in the control.

17. An analytical method including:
quantifying a miRNA selected from the group consisting of hsa-miR-126-3p, hsa-miR-326, hsa-miR-28-3p, hsa-miR-425-3p, hsa-miR-93-3p, hsa-miR-24-3p, hsa-miR-222-3p, hsa-miR-361-5p and hsa-miR-342-3p in a subject, and
determining a probability that the subject has breast, pancreatic or colorectal cancer.

18. The method of claim 17, further comprising quantifying the miRNA in a control,
wherein the determination is made by comparing a quantitative value of the miRNA in the subject with a quantitative value of the miRNA in the control to determine a probability that the subject has breast, pancreatic or colorectal cancer.

19. The method of claim 18, wherein the control is a healthy individual or an individual known to have at least one of breast, pancreatic or colorectal cancer.

20. The method of claim 19, wherein the control is a healthy individual, and wherein the determination includes determining that the subject has breast, pancreatic or colorectal cancer when a quantitative value of hsa-miR-126-3p, hsa-miR-326, hsa-miR-28-3p, hsa-miR-425-3p, hsa-miR-93-3p, hsa-miR-24-3p, hsa-miR-222-3p or hsa-miR-361-5p in the subject is less than a quantitative value in the control.

21. The method of claim 19, wherein the control is a healthy individual or breast cancer patient, and wherein the determination includes determining that the subject has pancreatic or colorectal cancer when a quantitative value of hsa-miR-342-3p in the subject is less than a quantitative value in the control.

22. An analytical method, including:
quantifying a miRNA selected from the group consisting of hsa-miR-126-3p, hsa-miR-326, hsa-miR-28-3p, hsa-miR-425-3p, hsa-miR-93-3p, hsa-miR-24-3p, hsa-miR-222-3p, hsa-miR-361-5p, hsa-miR-342-3p and hsa-miR-10b-5p in a subject, and
determining a possibility that the subject has colorectal cancer.

23. The method of claim 22, further comprising quantifying the miRNA in a control,
wherein the determination is made by comparing a quantitative value of the miRNA in the subject with a quantitative value of the miRNA in the control to determine a possibility that the subject has colorectal cancer.

24. The method of claim 23, wherein the control is an individual known to be at least one of a healthy individual, breast cancer patient or pancreatic cancer patient.

25. The method of claim 24, wherein the control is a breast cancer patient or a pancreatic cancer patient, and wherein the determination includes determining that the subject has colorectal cancer when a quantitative value of each of hsa-miR-126-3p, hsa-miR-326, hsa-miR-28-3p, hsa-miR-425-3p, hsa-miR-93-3p, hsa-miR-24-3p, hsa-miR-222-3p or hsa-miR-361-5p in the subject is less than a quantitative value in the control.

26. The method of claim 24, wherein the control is a pancreatic cancer patient, and wherein the determination includes determining that the subject has colorectal cancer when a quantitative value of hsa-miR-342-3p in the subject is less than a quantitative value in the control.

27. The method of claim 24, wherein the control is a healthy individual, a breast cancer patient, or a pancreatic cancer patient, and wherein the determination includes determining that the subject has colorectal cancer when a e quantitative value of hsa-miR-10b-5p in the subject is less than a quantitative value in the control.

28. The method of claim 1, wherein the quantification is performed using PCR, LAMP, or microarray.

29. The method of claim 1, wherein the sample is serum or plasma.

30. The method of claim 2, wherein the quantitative value of the miRNA in the subject and the quantitative value of the miRNA in the control are standardized by a standard miRNA commonly contained in the subject and the control.

31. The method of claim 30, wherein the standard miRNA is hsa-miR-486-5p.

32. A kit for determining the probability that the subject has breast, pancreatic, lung, gastric and colorectal cancer, comprising at least one nucleic acid set that specifically binds to a miRNA selected from the group consisting of hsa-miR-128-3p, hsa-miR-151a-3p, hsa-miR-143-3p, hsa-miR-4770, hsa-miR-1296-5p, hsa-miR-125a-5p, hsa-miR-409-3p, hsa-miR-99a-5p and hsa-miR-215-5p.

33. The kit of claim 32, wherein the nucleic acid that specifically binds to the miRNA is a reverse transcription primer for reverse transcription of the miRNA, an elongation primer for elongation of the miRNA, an amplification primer set for amplifying the miRNA, or a nucleic acid probe for detecting the miRNA.


SEQUENCE LISTING (133 sequences)

SEQ ID NO: 1 - RNA, 21 nt, Homo sapiens
  source 1..21: mol_type = transcribed RNA
  misc_feature 1..21: note = hsa-miR-128-3p
tcacagtgaaccggtctcttt

SEQ ID NO: 2 - RNA, 21 nt, Homo sapiens
  source 1..21: mol_type = transcribed RNA
  misc_feature 1..21: note = hsa-miR-151a-3p
ctagactgaagctccttgagg

SEQ ID NO: 3 - RNA, 21 nt, Homo sapiens
  source 1..21: mol_type = transcribed RNA
  misc_feature 1..21: note = hsa-miR-143-3p
tgagatgaagcactgtagctc

SEQ ID NO: 4 - RNA, 18 nt, Homo sapiens
  source 1..18: mol_type = transcribed RNA
  misc_feature 1..18: note = hsa-miR-4770
tgagatgacactgtagct

SEQ ID NO: 5 - RNA, 22 nt, Homo sapiens
  source 1..22: mol_type = transcribed RNA
  misc_feature 1..22: note = hsa-miR-1296-5p
ttagggccctggctccatctcc

SEQ ID NO: 6 - RNA, 24 nt, Homo sapiens
  source 1..24: mol_type = transcribed RNA
  misc_feature 1..24: note = hsa-miR-125a-5p
tccctgagaccctttaacctgtga

SEQ ID NO: 7 - RNA, 22 nt, Homo sapiens
  source 1..22: mol_type = transcribed RNA
  misc_feature 1..22: note = hsa-miR-409-3p
gaatgttgctcggtgaacccct

SEQ ID NO: 8 - RNA, 22 nt, Homo sapiens
  source 1..22: mol_type = transcribed RNA
  misc_feature 1..22: note = hsa-miR-99a-5p
aacccgtagatccgatcttgtg

SEQ ID NO: 9 - RNA, 21 nt, Homo sapiens
  source 1..21: mol_type = transcribed RNA
  misc_feature 1..21: note = hsa-miR-215-5p
atgacctatgaattgacagac

SEQ ID NO: 10 - RNA, 22 nt, Homo sapiens
  source 1..22: mol_type = transcribed RNA
  misc_feature 1..22: note = hsa-miR-126-3p
tcgtaccgtgagtaataatgcg

SEQ ID NO: 11 - RNA, 20 nt, Homo sapiens
  source 1..20: mol_type = transcribed RNA
  misc_feature 1..20: note = hsa-miR-326
cctctgggcccttcctccag

SEQ ID NO: 12 - RNA, 22 nt, Homo sapiens
  source 1..22: mol_type = transcribed RNA
  misc_feature 1..22: note = hsa-miR-28-3p
cactagattgtgagctcctgga

SEQ ID NO: 13 - RNA, 22 nt, Homo sapiens
  source 1..22: mol_type = transcribed RNA
  misc_feature 1..22: note = hsa-miR-425-3p
atcgggaatgtcgtgtccgccc

SEQ ID NO: 14 - RNA, 22 nt, Homo sapiens
  source 1..22: mol_type = transcribed RNA
  misc_feature 1..22: note = hsa-miR-93-3p
actgctgagctagcacttcccg

SEQ ID NO: 15 - RNA, 22 nt, Homo sapiens
  source 1..22: mol_type = transcribed RNA
  misc_feature 1..22: note = hsa-miR-24-3p
tggctcagttcagcaggaacag

SEQ ID NO: 16 - RNA, 21 nt, Homo sapiens
  source 1..21: mol_type = transcribed RNA
  misc_feature 1..21: note = hsa-miR-222-3p
agctacatctggctactgggt

SEQ ID NO: 17 - RNA, 22 nt, Homo sapiens
  source 1..22: mol_type = transcribed RNA
  misc_feature 1..22: note = hsa-miR-361-5p
ttatcagaatctccaggggtac

SEQ ID NO: 18 - RNA, 23 nt, Homo sapiens
  source 1..23: mol_type = transcribed RNA
  misc_feature 1..23: note = hsa-miR-342-3p
tctcacacagaaatcgcacccgt

SEQ ID NO: 19 - RNA, 23 nt, Homo sapiens
  source 1..23: mol_type = transcribed RNA
  misc_feature 1..23: note = hsa-miR-10b-5p
taccctgtagaaccgaatttgtg

SEQ ID NO: 20 - RNA, 21 nt, Homo sapiens
  source 1..21: mol_type = transcribed RNA
  misc_feature 1..21: note = hsa-miR-140-3p
taccacagggtagaaccacgg

SEQ ID NO: 21 - RNA, 21 nt, Homo sapiens
  source 1..21: mol_type = transcribed RNA
  misc_feature 1..21: note = hsa-miR-3666
cagtgcaagtgtagatgccga

SEQ ID NO: 22 - RNA, 21 nt, Homo sapiens
  source 1..21: mol_type = transcribed RNA
  misc_feature 1..21: note = hsa-miR-192-5p
ctgacctatgaattgacagcc

SEQ ID NO: 23 - RNA, 22 nt, Homo sapiens
  source 1..22: mol_type = transcribed RNA
  misc_feature 1..22: note = hsa-miR-4699-5p
agaagattgcagagtaagttcc

SEQ ID NO: 24 - RNA, 22 nt, Homo sapiens
  source 1..22: mol_type = transcribed RNA
  misc_feature 1..22: note = hsa-miR-3152-5p
attgcctctgttctaacacaag

SEQ ID NO: 25 - RNA, 22 nt, Homo sapiens
  source 1..22: mol_type = transcribed RNA
  misc_feature 1..22: note = hsa-miR-193b-5p
cggggttttgagggcgagatga

SEQ ID NO: 26 - RNA, 21 nt, Homo sapiens
  source 1..21: mol_type = transcribed RNA
  misc_feature 1..21: note = hsa-miR-5694
cagatcatgggactgtctcag

SEQ ID NO: 27 - RNA, 17 nt, Homo sapiens
  source 1..17: mol_type = transcribed RNA
  misc_feature 1..17: note = hsa-miR-3168
gagttctacagtcagac

SEQ ID NO: 28 - RNA, 22 nt, Homo sapiens
  source 1..22: mol_type = transcribed RNA
  misc_feature 1..22: note = hsa-miR-6755-3p
tgttgtcatgttttttccctag

SEQ ID NO: 29 - RNA, 21 nt, Homo sapiens
  source 1..21: mol_type = transcribed RNA
  misc_feature 1..21: note = hsa-miR-559
taaagtaaatatgcaccaaaa

SEQ ID NO: 30 - RNA, 22 nt, Homo sapiens
  source 1..22: mol_type = transcribed RNA
  misc_feature 1..22: note = hsa-miR-96-3p
aatcatgtgcagtgccaatatg

SEQ ID NO: 31 - RNA, 22 nt, Homo sapiens
  source 1..22: mol_type = transcribed RNA
  misc_feature 1..22: note = hsa-miR-876-3p
tggtggtttacaaagtaattca

SEQ ID NO: 32 - RNA, 20 nt, Homo sapiens
  source 1..20: mol_type = transcribed RNA
  misc_feature 1..20: note = hsa-miR-2052
tgttttgataacagtaatgt

SEQ ID NO: 33 - RNA, 23 nt, Homo sapiens
  source 1..23: mol_type = transcribed RNA
  misc_feature 1..23: note = hsa-miR-514a-5p
tactctggagagtgacaatcatg

SEQ ID NO: 34 - RNA, 22 nt, Homo sapiens
  source 1..22: mol_type = transcribed RNA
  misc_feature 1..22: note = hsa-let-7c-5p
tgaggtagtaggttgtatggtt

SEQ ID NO: 35 - RNA, 22 nt, Homo sapiens
  source 1..22: mol_type = transcribed RNA
  misc_feature 1..22: note = hsa-miR-196b-5p
taggtagtttcctgttgttggg

SEQ ID NO: 36 - RNA, 23 nt, Homo sapiens
  source 1..23: mol_type = transcribed RNA
  misc_feature 1..23: note = hsa-miR-1266-5p
cctcagggctgtagaacagggct

SEQ ID NO: 37 - RNA, 18 nt, Homo sapiens
  source 1..18: mol_type = transcribed RNA
  misc_feature 1..18: note = hsa-miR-4300
tgggagctggactacttc

SEQ ID NO: 38 - RNA, 21 nt, Homo sapiens
  source 1..21: mol_type = transcribed RNA
  misc_feature 1..21: note = hsa-miR-3151-5p
ggtggggcaatgggatcaggt

SEQ ID NO: 39 - RNA, 22 nt, Homo sapiens
  source 1..22: mol_type = transcribed RNA
  misc_feature 1..22: note = hsa-miR-5698
tgggggagtgcagtgattgtgg

SEQ ID NO: 40 - RNA, 23 nt, Homo sapiens
  source 1..23: mol_type = transcribed RNA
  misc_feature 1..23: note = hsa-miR-6756-5p
agggtggggctggaggtggggct

SEQ ID NO: 41 - RNA, 22 nt, Homo sapiens
  source 1..22: mol_type = transcribed RNA
  misc_feature 1..22: note = hsa-miR-615-5p
gggggtccccggtgctcggatc

SEQ ID NO: 42 - RNA, 22 nt, Homo sapiens
  source 1..22: mol_type = transcribed RNA
  misc_feature 1..22: note = hsa-miR-616-3p
agtcattggagggtttgagcag

SEQ ID NO: 43 - RNA, 20 nt, Homo sapiens
  source 1..20: mol_type = transcribed RNA
  misc_feature 1..20: note = hsa-miR-665
accaggaggctgaggcccct

SEQ ID NO: 44 - RNA, 21 nt, Homo sapiens
  source 1..21: mol_type = transcribed RNA
  misc_feature 1..21: note = hsa-miR-6765-3p
tcacctggctggcccgcccag

SEQ ID NO: 45 - RNA, 21 nt, Homo sapiens
  source 1..21: mol_type = transcribed RNA
  misc_feature 1..21: note = hsa-miR-6503-3p
gggactaggatgcagacctcc

SEQ ID NO: 46 - RNA, 21 nt, Homo sapiens
  source 1..21: mol_type = transcribed RNA
  misc_feature 1..21: note = hsa-miR-1237-5p
cgggggcggggccgaagcgcg

SEQ ID NO: 47 - RNA, 22 nt, Homo sapiens
  source 1..22: mol_type = transcribed RNA
  misc_feature 1..22: note = hsa-miR-206
tggaatgtaaggaagtgtgtgg

SEQ ID NO: 48 - RNA, 22 nt, Homo sapiens
  source 1..22: mol_type = transcribed RNA
  misc_feature 1..22: note = hsa-miR-744-5p
tgcggggctagggctaacagca

SEQ ID NO: 49 - RNA, 19 nt, Homo sapiens
  source 1..19: mol_type = transcribed RNA
  misc_feature 1..19: note = hsa-miR-6131
ggctggtcagatgggagtg

SEQ ID NO: 50 - RNA, 23 nt, Homo sapiens
  source 1..23: mol_type = transcribed RNA
  misc_feature 1..23: note = hsa-miR-4732-5p
tgtagagcagggagcaggaagct

SEQ ID NO: 51 - RNA, 22 nt, Homo sapiens
  source 1..22: mol_type = transcribed RNA
  misc_feature 1..22: note = hsa-miR-542-3p
tgtgacagattgataactgaaa

SEQ ID NO: 52 - RNA, 22 nt, Homo sapiens
  source 1..22: mol_type = transcribed RNA
  misc_feature 1..22: note = hsa-miR-127-3p
tcggatccgtctgagcttggct

SEQ ID NO: 53 - RNA, 19 nt, Homo sapiens
  source 1..19: mol_type = transcribed RNA
  misc_feature 1..19: note = hsa-miR-1246
aatggatttttggagcagg

SEQ ID NO: 54 - RNA, 22 nt, Homo sapiens
  source 1..22: mol_type = transcribed RNA
  misc_feature 1..22: note = hsa-miR-485-3p
gtcatacacggctctcctctct

SEQ ID NO: 55 - RNA, 22 nt, Homo sapiens
  source 1..22: mol_type = transcribed RNA
  misc_feature 1..22: note = hsa-miR-485-5p
agaggctggccgtgatgaattc

SEQ ID NO: 56 - RNA, 22 nt, Homo sapiens
  source 1..22: mol_type = transcribed RNA
  misc_feature 1..22: note = hsa-miR-5585-3p
ctgaatagctgggactacaggt

SEQ ID NO: 57 - RNA, 18 nt, Homo sapiens
  source 1..18: mol_type = transcribed RNA
  misc_feature 1..18: note = hsa-miR-4466
gggtgcgggccggcgggg

SEQ ID NO: 58 - RNA, 23 nt, Homo sapiens
  source 1..23: mol_type = transcribed RNA
  misc_difference 1..23: note = hsa-miR-19b-3p
tgtgcaaatccatgcaaaactga

SEQ ID NO: 59 - RNA, 21 nt, Homo sapiens
  source 1..21: mol_type = transcribed RNA
  misc_feature 1..21: note = hsa-miR-424-3p
caaaacgtgaggcgctgctat

SEQ ID NO: 60 - RNA, 22 nt, Homo sapiens
  source 1..22: mol_type = transcribed RNA
  misc_feature 1..22: note = hsa-miR-324-5p
cgcatcccctagggcattggtg

SEQ ID NO: 61 - RNA, 21 nt, Homo sapiens
  source 1..21: mol_type = transcribed RNA
  misc_feature 1..21: note = hsa-miR-4433b-5p
atgtcccacccccactcctgt

SEQ ID NO: 62 - RNA, 22 nt, Homo sapiens
  source 1..22: mol_type = transcribed RNA
  misc_feature 1..22: note = hsa-miR-150-5p
tctcccaacccttgtaccagtg

SEQ ID NO: 63 - RNA, 22 nt, Homo sapiens
  source 1..22: mol_type = transcribed RNA
  misc_feature 1..22: note = hsa-miR-146a-5p
tgagaactgaattccatgggtt

SEQ ID NO: 64 - RNA, 21 nt, Homo sapiens
  source 1..21: mol_type = transcribed RNA
  misc_feature 1..21: note = hsa-miR-483-3p
tcactcctctcctcccgtctt

SEQ ID NO: 65 - RNA, 22 nt, Homo sapiens
  source 1..22: mol_type = transcribed RNA
  misc_feature 1..22: note = hsa-miR-27b-5p
agagcttagctgattggtgaac

SEQ ID NO: 66 - RNA, 22 nt, Homo sapiens
  source 1..22: mol_type = transcribed RNA
  misc_feature 1..22: note = hsa-miR-493-3p
tgaaggtctactgtgtgccagg

SEQ ID NO: 67 - RNA, 21 nt, Homo sapiens
  source 1..21: mol_type = transcribed RNA
  misc_feature 1..21: note = hsa-miR-3150b-3p
tgaggagatcgtcgaggttgg

SEQ ID NO: 68 - RNA, 21 nt, Homo sapiens
  source 1..21: mol_type = transcribed RNA
  misc_feature 1..21: note = hsa-miR-652-3p
aatggcgccactagggttgtg

SEQ ID NO: 69 - RNA, 23 nt, Homo sapiens
  source 1..23: mol_type = transcribed RNA
  misc_feature 1..23: note = hsa-miR-9-5p
tctttggttatctagctgtatga

SEQ ID NO: 70 - RNA, 17 nt, Homo sapiens
  source 1..17: mol_type = transcribed RNA
  misc_feature 1..17: note = hsa-miR-3182
gcttctgtagtgtagtc

SEQ ID NO: 71 - RNA, 22 nt, Homo sapiens
  source 1..22: mol_type = transcribed RNA
  misc_feature 1..22: note = hsa-miR-4765
tgagtgattgatagctatgttc

SEQ ID NO: 72 - RNA, 22 nt, Homo sapiens
  source 1..22: mol_type = transcribed RNA
  misc_feature 1..22: note = hsa-miR-758-3p
tttgtgacctggtccactaacc

SEQ ID NO: 73 - RNA, 23 nt, Homo sapiens
  source 1..23: mol_type = transcribed RNA
  misc_feature 1..23: note = hsa-miR-1229-3p
ctctcaccactgccctcccacag

SEQ ID NO: 74 - RNA, 22 nt, Homo sapiens
  source 1..22: mol_type = transcribed RNA
  misc_feature 1..22: note = hsa-miR-15a-5p
tagcagcacataatggtttgtg

SEQ ID NO: 75 - RNA, 22 nt, Homo sapiens
  source 1..22: mol_type = transcribed RNA
  misc_feature 1..22: note = hsa-miR-21-5p
tagcttatcagactgatgttga

SEQ ID NO: 76 - RNA, 23 nt, Homo sapiens
  source 1..23: mol_type = transcribed RNA
  misc_feature 1..23: note = hsa-miR-767-5p
tgcaccatggttgtctgagcatg

SEQ ID NO: 77 - RNA, 22 nt, Homo sapiens
  source 1..22: mol_type = transcribed RNA
  misc_feature 1..22: note = hsa-miR-6797-3p
tgcatgacccttccctccccac

SEQ ID NO: 78 - RNA, 22 nt, Homo sapiens
  source 1..22: mol_type = transcribed RNA
  misc_feature 1..22: note = hsa-miR-32-5p
tattgcacattactaagttgca

SEQ ID NO: 79 - RNA, 23 nt, Homo sapiens
  source 1..23: mol_type = transcribed RNA
  misc_feature 1..23: note = hsa-miR-6830-3p
tgtctttcttctctcccttgcag

SEQ ID NO: 80 - RNA, 22 nt, Homo sapiens
  source 1..22: mol_type = transcribed RNA
  misc_feature 1..22: note = hsa-miR-576-3p
aagatgtggaaaaattggaatc

SEQ ID NO: 81 - RNA, 23 nt, Homo sapiens
  source 1..23: mol_type = transcribed RNA
  misc_feature 1..23: note = hsa-miR-6873-3p
ttctctctgtctttctctctcag

SEQ ID NO: 82 - RNA, 22 nt, Homo sapiens
  source 1..22: mol_type = transcribed RNA
  misc_feature 1..22: note = hsa-miR-1249-3p
acgcccttcccccccttcttca

SEQ ID NO: 83 - RNA, 22 nt, Homo sapiens
  source 1..22: mol_type = transcribed RNA
  misc_feature 1..22: note = hsa-miR-433-3p
atcatgatgggctcctcggtgt

SEQ ID NO: 84 - RNA, 21 nt, Homo sapiens
  source 1..21: mol_type = transcribed RNA
  misc_feature 1..21: note = hsa-miR-6886-3p
tgcccttctctcctcctgcct

SEQ ID NO: 85 - RNA, 23 nt, Homo sapiens
  source 1..23: mol_type = transcribed RNA
  misc_feature 1..23: note = hsa-miR-329-5p
gaggttttctgggtttctgtttc

SEQ ID NO: 86 - RNA, 23 nt, Homo sapiens
  source 1..23: mol_type = transcribed RNA
  misc_feature 1..23: note = hsa-miR-6859-5p
gagaggaacatgggctcaggaca

SEQ ID NO: 87 - RNA, 22 nt, Homo sapiens
  source 1..22: mol_type = transcribed RNA
  misc_feature 1..22: note = hsa-miR-15b-5p
tagcagcacatcatggtttaca

SEQ ID NO: 88 - RNA, 22 nt, Homo sapiens
  source 1..22: mol_type = transcribed RNA
  misc_feature 1..22: note = hsa-miR-25-3p
cattgcacttgtctcggtctga

SEQ ID NO: 89 - RNA, 22 nt, Homo sapiens
  source 1..22: mol_type = transcribed RNA
  misc_feature 1..22: note = hsa-miR-451a
aaaccgttaccattactgagtt

SEQ ID NO: 90 - RNA, 23 nt, Homo sapiens
  source 1..23: mol_type = transcribed RNA
  misc_feature 1..23: note = hsa-miR-425-5p
aatgacacgatcactcccgttga

SEQ ID NO: 91 - RNA, 23 nt, Homo sapiens
  source 1..23: mol_type = transcribed RNA
  misc_feature 1..23: note = hsa-miR-93-5p
caaagtgctgttcgtgcaggtag

SEQ ID NO: 92 - RNA, 22 nt, Homo sapiens
  source 1..22: mol_type = transcribed RNA
  misc_feature 1..22: note = hsa-miR-148a-3p
tcagtgcactacagaactttgt

SEQ ID NO: 93 - RNA, 23 nt, Homo sapiens
  source 1..23: mol_type = transcribed RNA
  misc_feature 1..23: note = hsa-miR-103a-3p
agcagcattgtacagggctatga

SEQ ID NO: 94 - RNA, 21 nt, Homo sapiens
  source 1..21: mol_type = transcribed RNA
  misc_feature 1..21: note = hsa-miR-3615
tctctcggctcctcgcggctc

SEQ ID NO: 95 - RNA, 23 nt, Homo sapiens
  source 1..23: mol_type = transcribed RNA
  misc_feature 1..23: note = hsa-miR-221-3p
agctacattgtctgctgggtttc

SEQ ID NO: 96 - RNA, 22 nt, Homo sapiens
  source 1..22: mol_type = transcribed RNA
  misc_feature 1..22: note = hsa-miR-484
tcaggctcagtcccctcccgat

SEQ ID NO: 97 - RNA, 22 nt, Homo sapiens
  source 1..22: mol_type = transcribed RNA
  misc_feature 1..22: note = hsa-miR-382-5p
gaagttgttcgtggtggattcg

SEQ ID NO: 98 - RNA, 21 nt, Homo sapiens
  source 1..21: mol_type = transcribed RNA
  misc_feature 1..21: note = hsa-miR-101-3p
tacagtactgtgataactgaa

SEQ ID NO: 99 - RNA, 22 nt, Homo sapiens
  source 1..22: mol_type = transcribed RNA
  misc_feature 1..22: note = hsa-miR-22-3p
aagctgccagttgaagaactgt

SEQ ID NO: 100 - RNA, 21 nt, Homo sapiens
  source 1..21: mol_type = transcribed RNA
  misc_feature 1..21: note = hsa-miR-27b-3p
ttcacagtggctaagttctgc

SEQ ID NO: 101 - RNA, 24 nt, Homo sapiens
  source 1..24: mol_type = transcribed RNA
  misc_feature 1..24: note = hsa-miR-155-5p
ttaatgctaatcgtgataggggtt

SEQ ID NO: 102 - RNA, 22 nt, Homo sapiens
  source 1..22: mol_type = transcribed RNA
  misc_feature 1..22: note = hsa-miR-196a-5p
taggtagtttcatgttgttggg

SEQ ID NO: 103 - RNA, 22 nt, Homo sapiens
  source 1..22: mol_type = transcribed RNA
  misc_feature 1..22: note = hsa-miR-22-5p
agttcttcagtggcaagcttta

SEQ ID NO: 104 - RNA, 23 nt, Homo sapiens
  source 1..23: mol_type = transcribed RNA
  misc_feature 1..23: note = hsa-miR-574-5p
tgagtgtgtgtgtgtgagtgtgt

SEQ ID NO: 105 - RNA, 22 nt, Homo sapiens
  source 1..22: mol_type = transcribed RNA
  misc_feature 1..22: note = hsa-miR-584-5p
ttatggtttgcctgggactgag

SEQ ID NO: 106 - RNA, 22 nt, Homo sapiens
  source 1..22: mol_type = transcribed RNA
  misc_feature 1..22: note = hsa-miR-99b-5p
cacccgtagaaccgaccttgcg

SEQ ID NO: 107 - RNA, 23 nt, Homo sapiens
  source 1..23: mol_type = transcribed RNA
  misc_feature 1..23: note = hsa-miR-664a-3p
tattcatttatccccagcctaca

SEQ ID NO: 108 - RNA, 23 nt, Homo sapiens
  source 1..23: mol_type = transcribed RNA
  misc_feature 1..23: note = hsa-miR-18a-5p
taaggtgcatctagtgcagatag

SEQ ID NO: 109 - RNA, 23 nt, Homo sapiens
  source 1..23: mol_type = transcribed RNA
  misc_feature 1..23: note = hsa-miR-181b-5p
aacattcattgctgtcggtgggt

SEQ ID NO: 110 - RNA, 22 nt, Homo sapiens
  source 1..22: mol_type = transcribed RNA
  misc_feature 1..22: note = hsa-miR-1-3p
tggaatgtaaagaagtatgtat

SEQ ID NO: 111 - RNA, 22 nt, Homo sapiens
  source 1..22: mol_type = transcribed RNA
  misc_feature 1..22: note = hsa-miR-660-5p
tacccattgcatatcggagttg

SEQ ID NO: 112 - RNA, 21 nt, Homo sapiens
  source 1..21: mol_type = transcribed RNA
  misc_feature 1..21: note = hsa-miR-27a-3p
ttcacagtggctaagttccgc

SEQ ID NO: 113 - RNA, 20 nt, Homo sapiens
  source 1..20: mol_type = transcribed RNA
  misc_feature 1..20: note = hsa-miR-1276
taaagagccctgtggagaca

SEQ ID NO: 114 - RNA, 22 nt, Homo sapiens
  source 1..22: mol_type = transcribed RNA
  misc_feature 1..22: note = hsa-miR-769-5p
tgagacctctgggttctgagct

SEQ ID NO: 115 - RNA, 22 nt, Homo sapiens
  source 1..22: mol_type = transcribed RNA
  misc_feature 1..22: note = hsa-miR-148b-3p
tcagtgcatcacagaactttgt

SEQ ID NO: 116 - RNA, 21 nt, Homo sapiens
  source 1..21: mol_type = transcribed RNA
  misc_feature 1..21: note = hsa-miR-5590-3p
aataaagttcatgtatggcaa

SEQ ID NO: 117 - RNA, 21 nt, Homo sapiens
  source 1..21: mol_type = transcribed RNA
  misc_feature 1..21: note = hsa-miR-3927-5p
gcctatcacatatctgcctgt

SEQ ID NO: 118 - RNA, 21 nt, Homo sapiens
  source 1..21: mol_type = transcribed RNA
  misc_feature 1..21: note = hsa-miR-1910-5p
ccagtcctgtgcctgccgcct

SEQ ID NO: 119 - RNA, 22 nt, Homo sapiens
  source 1..22: mol_type = transcribed RNA
  misc_feature 1..22: note = hsa-miR-509-3-5p
tactgcagacgtggcaatcatg

SEQ ID NO: 120 - RNA, 22 nt, Homo sapiens
  source 1..22: mol_type = transcribed RNA
  misc_feature 1..22: note = hsa-miR-338-5p
aacaatatcctggtgctgagtg

SEQ ID NO: 121 - RNA, 18 nt, Homo sapiens
  source 1..18: mol_type = transcribed RNA
  misc_feature 1..18: note = hsa-miR-513-5p
ttcacagggaggtgtcat

SEQ ID NO: 122 - RNA, 21 nt, Homo sapiens
  source 1..21: mol_type = transcribed RNA
  misc_feature 1..21: note = hsa-miR-520e-3p
aaagtgcttcctttttgaggg

SEQ ID NO: 123 - RNA, 23 nt, Homo sapiens
  source 1..23: mol_type = transcribed RNA
  misc_feature 1..23: note = hsa-miR-4657
aatgtggaagtggtctgaggcat

SEQ ID NO: 124 - RNA, 21 nt, Homo sapiens
  source 1..21: mol_type = transcribed RNA
  misc_feature 1..21: note = hsa-miR-3155a
ccaggctctgcagtgggaact

SEQ ID NO: 125 - RNA, 22 nt, Homo sapiens
  source 1..22: mol_type = transcribed RNA
  misc_feature 1..22: note = hsa-miR-132-3p
taacagtctacagccatggtcg

SEQ ID NO: 126 - RNA, 20 nt, Homo sapiens
  source 1..20: mol_type = transcribed RNA
  misc_feature 1..20: note = hsa-miR-6872-5p
tctcgcatcaggaggcaagg

SEQ ID NO: 127 - RNA, 22 nt, Homo sapiens
  source 1..22: mol_type = transcribed RNA
  misc_feature 1..22: note = hsa-miR-16-5p
tagcagcacgtaaatattggcg

SEQ ID NO: 128 - RNA, 22 nt, Homo sapiens
  source 1..22: mol_type = transcribed RNA
  misc_feature 1..22: note = hsa-let-7a-5p
tgaggtagtaggttgtatagtt

SEQ ID NO: 129 - RNA, 22 nt, Homo sapiens
  source 1..22: mol_type = transcribed RNA
  misc_feature 1..22: note = hsa-let-7i-5p
tgaggtagtagtttgtgctgtt

SEQ ID NO: 130 - RNA, 22 nt, Homo sapiens
  source 1..22: mol_type = transcribed RNA
  misc_feature 1..22: note = hsa-miR-30d-5p
tgtaaacatccccgactggaag

SEQ ID NO: 131 - RNA, 22 nt, Homo sapiens
  source 1..22: mol_type = transcribed RNA
  misc_feature 1..22: note = hsa-let-7f-5p
tgaggtagtagattgtatagtt

SEQ ID NO: 132 - RNA, 21 nt, Homo sapiens
  source 1..21: mol_type = transcribed RNA
  misc_feature 1..21: note = hsa-miR-3074-5p
gttcctgctgaactgagccag

SEQ ID NO: 133 - RNA, 22 nt, Homo sapiens
  source 1..22: mol_type = transcribed RNA
  misc_feature 1..22: note = hsa-miR-486-5p
tcctgtactgagctgccccgag